SEQUENCE LISTING (69 sequences)

SEQ ID NO: 1 - DNA, 6 bp, Artificial
  Description of the sequence generic CpG motif of formula 5′-X1X2CGX3X4-3′ (hexamer) (description p. 7)
  misc_feature (1)..(2): n = any naturally occurring nucleotide or an analogue thereof
  misc_feature (5)..(6): n = any naturally occurring nucleotide or an analogue thereof
nncgnn

SEQ ID NO: 2 - DNA, 8 bp, Artificial
  Description of the sequence generic CpG motif of formula 5′-X1X2X3CGX4X5X6-3′ (octamer) (description p. 7)
  misc_feature (1)..(3): n = any naturally occurring nucleotide or an analogue thereof
  misc_feature (6)..(8): n = any naturally occurring nucleotide or an analogue thereof
nnncgnnn

SEQ ID NO: 3 - DNA, 8 bp, Artificial
  Description of the sequence exemplary octamer sequence (description p. 8)
gacgttcc

SEQ ID NO: 4 - DNA, 8 bp, Artificial
  Description of the sequence exemplary octamer sequence (description p. 8)
gacgctcc

SEQ ID NO: 5 - DNA, 8 bp, Artificial
  Description of the sequence exemplary octamer sequence (description p. 8)
gacgtccc

SEQ ID NO: 6 - DNA, 8 bp, Artificial
  Description of the sequence exemplary octamer sequence (description p. 8)
gacgcccc

SEQ ID NO: 7 - DNA, 8 bp, Artificial
  Description of the sequence exemplary octamer sequence (description p. 8)
agcgttcc

SEQ ID NO: 8 - DNA, 8 bp, Artificial
  Description of the sequence exemplary octamer sequence (description p. 8)
agcgctcc

SEQ ID NO: 9 - DNA, 8 bp, Artificial
  Description of the sequence exemplary octamer sequence (description p. 8)
agcgtccc

SEQ ID NO: 10 - DNA, 8 bp, Artificial
  Description of the sequence exemplary octamer sequence (description p. 8)
agcgcccc

SEQ ID NO: 11 - DNA, 8 bp, Artificial
  Description of the sequence exemplary octamer sequence (description p. 8)
aacgttcc

SEQ ID NO: 12 - DNA, 8 bp, Artificial
  Description of the sequence exemplary octamer sequence (description p. 8)
aacgctcc

SEQ ID NO: 13 - DNA, 8 bp, Artificial
  Description of the sequence exemplary octamer sequence (description p. 8)
aacgtccc

SEQ ID NO: 14 - DNA, 8 bp, Artificial
  Description of the sequence exemplary octamer sequence (description p. 8)
aacgcccc

SEQ ID NO: 15 - DNA, 8 bp, Artificial
  Description of the sequence exemplary octamer sequence (description p. 8)
ggcgttcc

SEQ ID NO: 16 - DNA, 8 bp, Artificial
  Description of the sequence exemplary octamer sequence (description p. 8)
ggcgctcc

SEQ ID NO: 17 - DNA, 8 bp, Artificial
  Description of the sequence exemplary octamer sequence (description p. 8)
ggcgtccc

SEQ ID NO: 18 - DNA, 8 bp, Artificial
  Description of the sequence exemplary octamer sequence (description p. 8)
ggcgcccc

SEQ ID NO: 19 - DNA, 8 bp, Artificial
  Description of the sequence exemplary octamer sequence (description p. 8)
gacgttcg

SEQ ID NO: 20 - DNA, 8 bp, Artificial
  Description of the sequence exemplary octamer sequence (description p. 8)
gacgctcg

SEQ ID NO: 21 - DNA, 8 bp, Artificial
  Description of the sequence exemplary octamer sequence (description p. 8)
gacgtccg

SEQ ID NO: 22 - DNA, 8 bp, Artificial
  Description of the sequence exemplary octamer sequence (description p. 8)
gacgcccg

SEQ ID NO: 23 - DNA, 8 bp, Artificial
  Description of the sequence exemplary octamer sequence (description p. 8)
agcgttcg

SEQ ID NO: 24 - DNA, 8 bp, Artificial
  Description of the sequence exemplary octamer sequence (description p. 8)
agcgctcg

SEQ ID NO: 25 - DNA, 8 bp, Artificial
  Description of the sequence exemplary octamer sequence (description p. 8)
agcgtccg

SEQ ID NO: 26 - DNA, 8 bp, Artificial
  Description of the sequence exemplary octamer sequence (description p. 8)
agcgcccg

SEQ ID NO: 27 - DNA, 8 bp, Artificial
  Description of the sequence exemplary octamer sequence (description p. 8)
aacgttcg

SEQ ID NO: 28 - DNA, 8 bp, Artificial
  Description of the sequence exemplary octamer sequence (description p. 8)
aacgctcg

SEQ ID NO: 29 - DNA, 8 bp, Artificial
  Description of the sequence exemplary octamer sequence (description p. 8)
aacgtccg

SEQ ID NO: 30 - DNA, 8 bp, Artificial
  Description of the sequence exemplary octamer sequence (description p. 8)
aacgcccg

SEQ ID NO: 31 - DNA, 8 bp, Artificial
  Description of the sequence exemplary octamer sequence (description p. 8)
ggcgttcg

SEQ ID NO: 32 - DNA, 8 bp, Artificial
  Description of the sequence exemplary octamer sequence (description p. 8)
ggcgctcg

SEQ ID NO: 33 - DNA, 8 bp, Artificial
  Description of the sequence exemplary octamer sequence (description p. 8)
ggcgtccg

SEQ ID NO: 34 - DNA, 8 bp, Artificial
  Description of the sequence exemplary octamer sequence (description p. 8)
ggcgcccg

SEQ ID NO: 35 - DNA, 22 bp, Artificial
  Description of the sequence exemplary RNA or DNA homopolymer (see description p. 10)
aaaaaaaaaaaaaaaaaaaaaa

SEQ ID NO: 36 - DNA, 22 bp, Artificial
  Description of the sequence exemplary RNA or DNA homopolymer (see description p. 10)
uuuuuuuuuuuuuuuuuuuuuu

SEQ ID NO: 37 - DNA, 22 bp, Artificial
  Description of the sequence exemplary RNA or DNA homopolymer (see description p. 10)
gggggggggggggggggggggg

SEQ ID NO: 38 - DNA, 22 bp, Artificial
  Description of the sequence exemplary RNA or DNA homopolymer (see description p. 10)
cccccccccccccccccccccc

SEQ ID NO: 39 - DNA, 22 bp, Artificial
  Description of the sequence exemplary RNA or DNA homopolymer (see description p. 10)
tttttttttttttttttttttt

SEQ ID NO: 40 - DNA, 20 bp, Artificial
  Description of the sequence exemplary RNA/DNA sequence contained in the lipid-modified nucleic acid (see description p. 12-13)
gcccgucuguugugugacuc

SEQ ID NO: 41 - DNA, 20 bp, Artificial
  Description of the sequence exemplary RNA/DNA sequence contained in the lipid-modified nucleic acid (see description p. 12-13)
gguaaguguaagguguaagg

SEQ ID NO: 42 - DNA, 20 bp, Artificial
  Description of the sequence exemplary RNA/DNA sequence contained in the lipid-modified nucleic acid (see description p. 12-13)
aauggauauggaauauggaa

SEQ ID NO: 43 - DNA, 20 bp, Artificial
  Description of the sequence exemplary RNA/DNA sequence contained in the lipid-modified nucleic acid (see description p. 12-13)
uccaugacguuccugacguu

SEQ ID NO: 44 - DNA, 20 bp, Artificial
  Description of the sequence exemplary RNA/DNA sequence contained in the lipid-modified nucleic acid (see description p. 12-13)
uccaggacuucucucagguu

SEQ ID NO: 45 - DNA, 20 bp, Artificial
  Description of the sequence exemplary RNA/DNA sequence contained in the lipid-modified nucleic acid (see description p. 12-13)
uccaugacguuccugaugcu

SEQ ID NO: 46 - DNA, 20 bp, Artificial
  Description of the sequence exemplary RNA/DNA sequence /contained in the lipid-modified nucleic acid (see description p. 12-13)
gcccgucuguugugugacuc

SEQ ID NO: 47 - DNA, 20 bp, Artificial
  Description of the sequence exemplary RNA/DNA sequence contained in the lipid-modified nucleic acid (see description p. 12-13)
gguaaguguaagguguaagg

SEQ ID NO: 48 - DNA, 20 bp, Artificial
  Description of the sequence exemplary RNA/DNA sequence contained in the lipid-modified nucleic acid (see description p. 12-13)
aauggauauggaauauggaa

SEQ ID NO: 49 - DNA, 21 bp, Artificial
  Description of the sequence exemplary RNA/DNA sequence contained in the lipid-modified nucleic acid (see description p. 12-13)
cucuggaggaaaagaaagutt

SEQ ID NO: 50 - DNA, 21 bp, Artificial
  Description of the sequence exemplary RNA/DNA sequence contained in the lipid-modified nucleic acid (see description p. 12-13)
caaugcaacucgcuucucgtt

SEQ ID NO: 51 - DNA, 19 bp, Artificial
  Description of the sequence exemplary RNA/DNA sequence contained in the lipid-modified nucleic acid (see description p. 12-13)
agcuuaaccuguccuucaa

SEQ ID NO: 52 - DNA, 19 bp, Artificial
  Description of the sequence exemplary RNA/DNA sequence contained in the lipid-modified nucleic acid (see description p. 12-13)
aaaaaaaacuguccuucaa

SEQ ID NO: 53 - DNA, 19 bp, Artificial
  Description of the sequence exemplary RNA/DNA sequence contained in the lipid-modified nucleic acid (see description p. 12-13)
aaaaaaaaauguccuucaa

SEQ ID NO: 54 - DNA, 19 bp, Artificial
  Description of the sequence exemplary RNA/DNA sequence contained in the lipid-modified nucleic acid (see description p. 12-13)
aaaaaaaaaaguccuucaa

SEQ ID NO: 55 - DNA, 19 bp, Artificial
  Description of the sequence exemplary RNA/DNA sequence contained in the lipid-modified nucleic acid (see description p. 12-13)
aaaaaaaaaaauccuucaa

SEQ ID NO: 56 - DNA, 19 bp, Artificial
  Description of the sequence exemplary RNA/DNA sequence contained in the lipid-modified nucleic acid (see description p. 12-13)
aaaaaaaaaaaaccuucaa

SEQ ID NO: 57 - DNA, 19 bp, Artificial
  Description of the sequence exemplary RNA/DNA sequence contained in the lipid-modified nucleic acid (see description p. 12-13)
agcuuaaccuguccuuaaa

SEQ ID NO: 58 - DNA, 19 bp, Artificial
  Description of the sequence exemplary RNA/DNA sequence contained in the lipid-modified nucleic acid (see description p. 12-13)
agcuuaaccuguccuaaaa

SEQ ID NO: 59 - DNA, 19 bp, Artificial
  Description of the sequence exemplary RNA/DNA sequence contained in the lipid-modified nucleic acid (see description p. 12-13)
agcuuaaccuguccaaaaa

SEQ ID NO: 60 - DNA, 19 bp, Artificial
  Description of the sequence exemplary RNA/DNA sequence contained in the lipid-modified nucleic acid (see description p. 12-13)
agcuuaaccugaaaaaaaa

SEQ ID NO: 61 - DNA, 20 bp, Artificial
  Description of the sequence exemplary RNA/DNA sequence contained in the lipid-modified nucleic acid (see description p. 12-13)
uguccuucaauguccuucaa

SEQ ID NO: 62 - DNA, 19 bp, Artificial
  Description of the sequence exemplary RNA/DNA sequence contained in the lipid-modified nucleic acid (see description p. 12-13)
agcuuaaccuguccuucau

SEQ ID NO: 63 - DNA, 19 bp, Artificial
  Description of the sequence exemplary RNA/DNA sequence contained in the lipid-modified nucleic acid (see description p. 12-13)
agcuuaaccuguccuucuu

SEQ ID NO: 64 - DNA, 24 bp, Artificial
  Description of the sequence exemplary RNA/DNA sequence contained in the lipid-modified nucleic acid (see description p. 12-13)
agcuuaaccuguccuucaacuaca

SEQ ID NO: 65 - DNA, 23 bp, Artificial
  Description of the sequence exemplary RNA/DNA sequence contained in the lipid-modified nucleic acid (see description p. 12-13)
caaauugaaggacagguuaagcu

SEQ ID NO: 66 - DNA, 16 bp, Artificial
  Description of the sequence exemplary RNA/DNA sequence contained in the lipid-modified nucleic acid (see description p. 12-13)
uuaaccuguccuucaa

SEQ ID NO: 67 - DNA, 13 bp, Artificial
  Description of the sequence exemplary RNA/DNA sequence contained in the lipid-modified nucleic acid (see description p. 12-13)
aaccuguccuuca

SEQ ID NO: 68 - RNA, 13 nt, Artificial
  Description of the sequence Koszak sequence (see description p. 31)
gccgccaccaugg

SEQ ID NO: 69 - RNA, 15 nt, Artificial
  Description of the sequence generic sequence of a stabilising sequence (see description p. 32)
  misc_feature (1)..(1): n = C or U
  misc_feature (5)..(5): n = any naturally occurring nucleotide or an analogue thereof
  repeat_unit (5)..(5): x = as desired
  misc_feature (9)..(9): n = U or A
  repeat_unit (10)..(10): x = as desired
  modified_base (10)..(10): n = pyrimidine
  misc_feature (13)..(13): n = C or U
nccancccnnucncc

(19) United States
(12) Patent Application Publication
(10) Pub. No.: US 2007/0280929 A1
(43) Pub. Date: Dec. 6, 2007

(54) ADJUVANT IN THE FORM OF A LIPID-MODIFIED NUCLEIC ACID

(75) Inventors: Ingmar Hoerr, Tubingen (DE); Thomas Ketterer, Tubingen (DE); Steve Pascolo, Tubingen (DE)

Correspondence Address: CONNOLLY BOVE LODGE & HUTZ LLP, 1875 EYE STREET, N.W., SUITE 1100, WASHINGTON, DC, 20036, US

(73) Assignee: CUREVAC GMBH, Tubingen (DE)

(21) Appl. No.: 11/748,181

(22) Filed: May 14, 2007

Related U.S. Application Data
(63) Continuation-in-part of application No. PCT/EP06/08321, filed on Aug. 24, 2006.

(30) Foreign Application Priority Data
Feb. 17, 2006 (DE) 10 2006 007 433.5

(51) Int. Cl. A61K 31/7088; A61K 38/00; A61K 39/00; A61K 39/395; A61P 31/00; A61P 31/16; A61P 31/18; A61P 35/00; A61P 35/02; C07H 21/00

(52) U.S. Cl. 424/130.1; 424/184.1; 514/2; 514/44; 536/23.1

(57) ABSTRACT

The present invention relates to an immune-stimulating adjuvant in the form of a lipid-modified nucleic acid, optionally in combination with further adjuvants. The invention relates further to a pharmaceutical composition and to a vaccine, each containing an immune-stimulating adjuvant according to the invention, at least one active ingredient and optionally a pharmaceutically acceptable carrier and/or further auxiliary substances and additives and/or further adjuvants. The present invention relates likewise to the use of the pharmaceutical composition according to the invention and of the vaccine according to the invention for the treatment of infectious diseases or cancer diseases. Likewise, the present invention includes the use of the immune-stimulating adjuvant according to the invention in the preparation of a pharmaceutical composition for the treatment of cancer diseases or infectious diseases.

### CROSS REFERENCE TO RELATED APPLICATIONS

This application claims priority to PCT/EP2006/008321, filed Aug. 24, 2006, which claims priority to DE 10 2006 007 433.5, filed Feb. 17, 2006, the entire contents of both of which are specifically incorporated herein by reference.

### FIELD OF THE INVENTION

The present invention relates to an immune-stimulating adjuvant in the form of a lipid-modified nucleic acid, optionally in combination with further adjuvants. The invention relates further to a pharmaceutical composition and to a vaccine, each containing an immune-stimulating adjuvant according to the invention, at least one active ingredient and optionally a pharmaceutically acceptable carrier and/or further auxiliary substances and additives and/or further adjuvants. The present invention relates likewise to the use of the pharmaceutical composition according to the invention and of the vaccine according to the invention for the treatment of infectious diseases or cancer diseases. Likewise, the present invention includes the use of the immune-stimulating adjuvant according to the invention in the preparation of a pharmaceutical composition for the treatment of cancer diseases or infectious diseases.

### BACKGROUND OF THE INVENTION

In both conventional and genetic vaccination, the problem frequently occurs that only a small and therefore frequently inadequate immune response is brought about in the organism to be treated or inoculated. For this reason there are frequently added to vaccines or active ingredients so-called adjuvants, that is to say substances or compositions that are able to increase and/or influence in a directed manner an immune response, for example to an antigen. For example, it is known that the effectiveness of some injectable medicinal active ingredients can be improved significantly by combining the active ingredient with an adjuvant which is capable of influencing the release of the active ingredient into the host cell system and optionally its uptake into the host cells. In this manner it is possible to achieve an effect that is comparable to the periodic administration of many small doses at regular intervals. The term “adjuvant” conventionally refers in this context to a compound or composition that serves as binder, carrier or auxiliary substance for immunogens and/or other pharmaceutically active compounds.

A number of compounds and compositions have been proposed as adjuvants in the prior art, for example Freund's adjuvant, metal oxides (aluminium hydroxide, etc.), alum, inorganic chelates or salts thereof, various paraffin-like oils, synthetic resins, alginates, mucoids, polysaccharide compounds, caseinates, as well as compounds isolated from blood and/or blood clots, such as, for example, fibrin derivatives, etc. However, such adjuvants in most cases produce undesirable side-effects, for example very painful irritation and inflammation at the site of administration. Furthermore, toxic side-effects, in particular tissue necroses, are also observed. Finally, these known adjuvants in most cases bring about only inadequate stimulation of the cellular immune response, because only B-cells are activated.

For example, alums, metal oxides and chelates of salts have been associated with the generation of sterile abscesses. In addition, there are doubts among scientific experts that such compounds are excreted again fully. It is assumed, rather, that they result in undesirable inorganic residues in the body. Although such compounds usually have low toxicity, it is possible for them to be phagocyted by the cells of the reticulo-endothelial system (littoral and sinusoidal cells of the liver and spleen) as part of the insoluble debris. Furthermore, there are indications that such debris can have a damaging effect on the various filter mechanisms of the body, for example the kidneys, the liver or the spleen. Such residues accordingly represent a latent, ever present source of risk in the body and, generally, for the immune system.

The synthetic oils and petroleum derivatives used as adjuvants in the prior art likewise lead to adverse effects. However, these compounds are undesirable in particular because they metabolise rapidly in the body and decompose into their aromatic hydrocarbon compounds. It is known, however, that such aromatic hydrocarbon compounds can have a carcinogenic action to the greatest degree. Moreover, it has been demonstrated that such compounds are likewise associated with the formation of sterile abscesses and can rarely be removed from the body again completely.

Compounds isolated from animals, such as, for example, gelatin, are also frequently unsuitable as adjuvants for the purpose of immune stimulation. Although such compounds do not usually have a destructive action on the host organism or the host cells in question, they typically migrate too rapidly from the injection site into the host organism or into the host cells, so that the properties generally desired for an adjuvant, such as, for example, delayed release of an active ingredient optionally injected together with the adjuvant, etc., are seldom achieved. Such rapid distribution can in some cases be counteracted with tannins or other (inorganic) compounds. The metabolism of such additional compounds and their whereabouts in the body have not been fully explained, however. In this case too, therefore, it is reasonable to assume that these compounds accumulate in the debris and thus considerably interfere with the filtration mechanisms, for example the kidney, liver and/or spleen cells. Also, the property of gelatin of swelling when administered parenterally can lead under in vivo conditions to unpleasant side-effects, such as, for example, swelling, in particular at the site of administration, and to a feeling of illness.

In the case of compounds isolated from blood and/or blood clots, such as, for example, fibrin derivatives, etc., immune-stimulating effects have typically been demonstrated. However, most of these compounds, when present as adjuvants, are unsuitable because of their side-effects on the immune system (which occur in parallel with the desired immunogenic properties). For example, many of these compounds are categorised as allergenic and in some circumstances bring about an over-reaction of the immune system which far exceeds the desired degree. These compounds are therefore likewise unsuitable as adjuvants for immune stimulation for the mentioned reasons.

Immune responses can additionally be produced directly using nucleic acids as adjuvant. For example, DNA plays a central role in the production of immune responses. Bacterial DNA, for example, is known to have an immune-stimulating action owing to the presence of unmethylated CG motifs, and such CpG-DNA has therefore been proposed as an immune-stimulating agent and as an adjuvant for vaccines (see U.S. Pat. No. 5,663,153). This immune-stimulating property of DNA can also be achieved by DNA oligonucleotides which are stabilised by phosphorothioate modification (U.S. Pat. No. 6,239,116). Finally, U.S. Pat. No. 6,406,705 discloses adjuvant compositions which contain a synergistic combination of a CpG oligodeoxyribonucleotide and a non-nucleic acid adjuvant.

However, the use of DNA as adjuvant can be less advantageous from several points of view. DNA is decomposed only relatively slowly in the bloodstream, so that, when immune-stimulating (foreign) DNA is used, the formation of anti-DNA antibodies can occur, which has been confirmed in an animal model in the mouse (Gilkeson et al., J. Clin. Invest. 1995, 95: 1398-1402). The possible persistence of (foreign) DNA in the organism can thus lead to over-activation of the immune system, which is known to result in mice in splenomegaly (Montheith et al., Anticancer Drug Res. 1997, 12(5): 421-432). Furthermore, (foreign) DNA can interact with the host genome and cause mutations, in particular by integration into the host genome. For example, insertion of the introduced (foreign) DNA into an intact gene can occur, which represents a mutation which can impede or even eliminate completely the function of the endogenous gene. As a result of such integration events, on the one hand enzyme systems that are vital to the cell can be destroyed, and on the other hand there is also a risk that the cell so changed will be transformed into a degenerate state if, by the integration of the (foreign) DNA, a gene that is critical for the regulation of cell growth is changed. Therefore, in processes known hitherto, a possible risk of cancer formation cannot be ruled out when using (foreign) DNA as immune-stimulating agent.

It is therefore generally more advantageous to use RNA as adjuvant for producing such immune responses, because RNA has a substantially shorter half-life in vivo than DNA. Nevertheless, even the use of RNA as adjuvant has limitations. For example, RNA sequences disclosed hitherto in the prior art exhibit only limited cell permeability in vivo. This can in turn require an increased amount of RNA for immune stimulation, which, regardless of the increased costs owing to the increased amounts of RNA to be administered, involves the risk of the mostly undesirable side-effects described generally hereinbefore, for example very painful irritation and inflammation at the site of administration. Also, toxic side-effects cannot be ruled out when large amounts of the immune-stimulating agent are administered.

Despite the successes demonstrated hitherto, there is therefore an increased need for, and considerable interest in, improved immune stimulation, in particular agents that on the one hand are suitable for triggering an efficient immune response in the patient to be treated or inoculated and on the other hand effectively assist the uptake into the body or body cells of an active ingredient that may optionally additionally be present.

### SUMMARY OF THE INVENTION

This object is achieved by an immune-stimulating adjuvant according to the invention in the form of a lipid-modified nucleic acid. This lipid-modified nucleic acid consists according to the invention of a nucleic acid, at least one linker covalently linked with that nucleic acid, and at least one lipid covalently linked with the respective linker. Alternatively, the lipid-modified nucleic acid consists according to the invention of a (at least one) nucleic acid and at least one (bifunctional) lipid covalently linked with that nucleic acid (without a linker). According to a third alternative, the lipid-modified nucleic acid consists according to the invention of a nucleic acid, at least one linker covalently linked with that nucleic acid, and at least one lipid covalently linked with the respective linker, and also at least one (bifunctional) lipid covalently linked with that nucleic acid (without a linker).

A lipid-modified nucleic acid of the invention may comprise a nucleic acid, at least one linker covalently linked with that nucleic acid, and at least one lipid covalently linked with the respective linker. Alternatively, a lipid-modified nucleic acid of the invention may comprise a (at least one) nucleic acid and at least one (bifunctional) lipid covalently linked with that nucleic acid (without a linker). According to another alternative, a lipid-modified nucleic acid of the invention may comprise a nucleic acid, at least one linker covalently linked with that nucleic acid, and at least one lipid covalently linked with the respective linker, and also at least one (bifunctional) lipid covalently linked with that nucleic acid (without a linker).

In some embodiments, the present invention provides an immune-stimulating adjuvant. An immune-stimulating adjuvant of the invention may comprise a lipid-modified nucleic acid. In some embodiments, an immune-stimulating adjuvant of the invention may comprise a lipid-modified nucleic acid that comprises a nucleic acid covalently attached to a linker and a lipid covalently attached to the linker. In some embodiments, an immune-stimulating adjuvant may comprise a lipid-modified nucleic acid that comprises at least one nucleic acid and at least one bifunctional lipid covalently linked to the nucleic acid. In some embodiments, an immune-stimulating adjuvant may comprise a lipid-modified nucleic acid that comprises a nucleic acid, at least one linker covalently linked to the nucleic acid, at least one lipid covalently linked to the linker, and at least one bifunctional lipid covalently linked to the nucleic acid. In some embodiments, an immune-stimulating adjuvant may comprise a lipid-modified nucleic acid that comprises a nucleic acid covalently attached to a linker and contains 3 to 8 lipids per nucleic acid, wherein at least one lipid is covalently linked with the linker. In some embodiments, an immune-stimulating adjuvant may comprise a lipid-modified nucleic acid that comprises a nucleic acid covalently attached to a linker and contains 3 to 8 lipids per nucleic acid and wherein all of the lipids are covalently linked with the linker In some embodiments, an immune-stimulating adjuvant may comprise a lipid-modified nucleic acid that contains 3 to 8 lipids per nucleic acid and the lipids may be covalently linked directly with the nucleic acid. In some embodiments, an immune-stimulating adjuvant may comprise a lipid-modified nucleic acid that is selected from a group consisting of RNA, DNA, an RNA oligonucleotide, a DNA oligonucleotide, an RNA homopolymer, a DNA homopolymer or a CpG nucleic acid. In some embodiments, an immune-stimulating adjuvant may comprise a lipid-modified nucleic acid that is selected from a group consisting of a single-stranded nucleic acid, a double-stranded nucleic acid, a homoduplex nucleic acid, a heteroduplex nucleic acid, a linear nucleic acid, and a circular nucleic acid. In some embodiments, an immune-stimulating adjuvant may comprise a lipid-modified nucleic acid that has a length selected from the group consisting of from approximately 2 to approximately 1000 nucleotides, from approximately 5 to approximately 200 nucleotides, from approximately 6 to approximately 100 nucleotides, from approximately 6 to approximately 40 nucleotides, and from approximately 6 to approximately 31 nucleotides. In some embodiments, an immune-stimulating adjuvant may comprise a lipid-modified nucleic acid that comprises a sequence selected from the group consisting of SEQ ID NOs: 1-67. In some embodiments, an immune-stimulating adjuvant may comprise a lipid-modified nucleic acid that comprises a sequence that is at least 60% identical with a sequence selected from the group consisting of SEQ ID NOs: 1-67. In some embodiments, an immune-stimulating adjuvant may comprise a lipid-modified nucleic acid that comprises at least one lipid is selected from the group consisting of vitamins, α-tocopherol (vitamin E), RRR-α-tocopherol (D-α-tocopherol), L-α-tocopherol, racemate D,L-α-tocopherol, vitamin A, derivatives of vitamin A, retinoic acid, retinol, vitamin D, derivatives of vitamin D, ergosterol precursors of vitamin D, vitamin E, derivatives of vitamin E, vitamin E succinate (VES), vitamin K, derivatives of vitamin K, quinone compounds, phytol compounds, steroids, bile acids, cholic acid, deoxycholic acid, dehydrocholic acid, cortisone, digoxygenin, testosterone, cholesterol, thiocholesterol, polyalkylene glycols, aliphatic groups, C1-C20-alkanes, C1-C20-alkenes, C1-C20-alkanols, dodecanediol, hexadecanol, undecyl radicals, phospholipids, phosphatidylglycerol, diacylphosphatidylglycerol, phosphatidylcholine, dipalmitoylphosphatidylcholine, distearoylphosphatidylcholine, phosphatidylserine, phosphatidylethanolamine, di-hexadecyl-rac-glycerol, sphingolipids, cerebrosides, gangliosides, triethylammonium 1,2-di-O-hexadecyl-rac-glycero-3-H-phosphonate, polyamines, polyalkylene glycols, polyethylene glycol (PEG), hexaethylene glycol (HEG), palmitin, palmityl radicals, octadecylamines, hexylamino-carbonyl-oxycholesterol radicals, waxes, terpenes, alicyclic hydrocarbons, saturated fatty acid radicals, or mono-unsaturated fatty acid radicals, or poly-unsaturated fatty acid radicals. In some embodiments, an immune-stimulating adjuvant may comprise a lipid-modified nucleic acid comprising a linker that may contain 2-4 reactive groups. In such cases the reactive groups may be independently selected from the group consisting of a hydroxy group, an amino group and an alkoxy group. In some embodiments, an immune-stimulating adjuvant may comprise a lipid-modified nucleic acid comprising a linker that is selected from the group consisting of glycol, glycerol, glycerol derivatives, 2-aminobutyl-1,3-propanediol, 2-aminobutyl-1,3-propanediol derivatives, a 2-aminobutyl-1,3-propanediol scaffold, pyrrolidine linkers, and pyrrolidine-containing organic molecules. In some embodiments, an immune-stimulating adjuvant may comprise a lipid-modified nucleic acid comprising a nucleic acid that comprises a 3′ end and a 5′ end and the nucleic acid may be modified with lipid at the 3′-end, the 5′-end or at both the 3′-end and the 5′-end. In some embodiments, an immune-stimulating adjuvant may comprise a lipid-modified nucleic acid comprising a nucleic acid that comprises a 3′ end and a 5′ end and the nucleic acid may may comprise a linker that may be attached to the nucleic acid at the 3′-end, the 5′-end or at both the 3′-end and the 5′-end. In some embodiments, an immune-stimulating adjuvant may comprise a lipid-modified nucleic acid comprising a nucleic acid that comprises at least one chemical modification. In some embodiments, an immune-stimulating adjuvant may comprise a lipid-modified nucleic acid comprising a nucleic acid that is RNA comprising a 5′-end and a 3′-end and comprises a 5′-end a cap structure, a 3′-end poly-A tail, or both a 5′-end a cap structure and a 3′-end poly-A tail. In some embodiments, an immune-stimulating adjuvant may comprise a lipid-modified nucleic acid may be used in conjunction with an adjuvant selected from the group consisting of aluminium hydroxide, complete Freund's adjuvant, incomplete Freund's adjuvant, stabilising cationic peptides, polypeptides, protamine, nucleoline, spermine, spermidine, cationic polysaccharides, chitosan, TDM, MDP, muramyl dipeptide, alum solution, pluronics, lipopeptides, and Pam3Cys.

In some embodiments, the present invention provides a pharmaceutical composition comprising an immune-stimulating adjuvant comprising a lipid-modified nucleic acid and at least one active ingredient. In some embodiments, a pharmaceutical composition of the invention may further comprise at least one ingredient selected from the group consisting of pharmaceutically acceptable carriers, pharmaceutically acceptable additives and adjuvants. Any active ingredient known to those skilled in the art may be used. In some embodiments, an active ingredient may be selected from peptides, proteins, nucleic acids, low molecular weight organic or inorganic compounds having a molecular weight less than 5000, sugars, antigens, antibodies, and therapeutic agents. A pharmaceutical composition of the invention may further comprise an adjuvant selected from the group consisting of aluminium hydroxide, complete Freund's adjuvant, incomplete Freund's adjuvant, stabilising cationic peptides, polypeptides, protamine, nucleoline, spermine, spermidine, cationic polysaccharides, chitosan, TDM, MDP, muramyl dipeptide, alum solution, pluronics, lipopeptides, and Pam3Cys. Pharmaceutical compositions of the invention may be used for any purpose known to those skilled in the art. In some embodiments, a pharmaceutical composition according to of the invention may be a vaccine.

In some embodiments, the present invention provides methods of treating a subject (e.g., a mammal such as a human) in need thereof by administering an immune-stimulating adjuvant according to of the invention. Such methods of treating may further comprise administering a therapeutic agent. Methods of the invention may be used to treat any disease known to those skilled in the art, for example, cancer and/or infectious disease. Examples of diseases that may be treated according to the invention include, but are not limited to, colon carcinomas, melanomas, renal carcinomas, lymphomas, acute myeloid leukaemia (AML), acute lymphoid leukaemia (ALL), chronic myeloid leukaemia (CML), chronic lymphocytic leukaemia (CLL), gastrointestinal tumours, pulmonary carcinomas, gliomas, thyroid tumours, mammary carcinomas, prostate tumours, hepatomas, virus-induced tumours, papilloma virus-induced carcinomas, cervical carcinoma, adenocarcinomas, herpes virus-induced tumours, Burkitt's lymphoma, EBV-induced B-cell lymphoma, heptatitis B-induced tumours, hepatocell carcinoma, HTLV-1-induced lymphomas, HTLV-2-induced lymphomas, acoustic neuromas, cervical cancer, lung cancer, pharyngeal cancer, anal carcinomas, glioblastomas, lymphomas, rectal carcinomas, astrocytomas, brain tumours, stomach cancer, retinoblastomas, basaliomas, brain metastases, medulloblastomas, vaginal cancer, pancreatic cancer, testicular cancer, melanomas, thyroidal carcinomas, bladder cancer, Hodgkin's syndrome, meningiomas, Schneeberger disease, bronchial carcinomas, hypophysis tumour, Mycosis fungoides, esophageal cancer, breast cancer, carcinoids, neurinomas, spinaliomas, laryngeal cancer, renal cancer, thymomas, corpus carcinomas, bone cancer, non-Hodgkin's lymphomas, urethral cancer, CUP syndrome, head tumors, neck tumours, oligodendrogliomas, vulval cancer, intestinal cancer, colon carcinomas, esophageal carcinomas, warts, tumours of the small intestine, craniopharyngeomas, ovarian carcinomas, genital tumours, ovarian cancer, liver cancer, pancreatic carcinomas, cervical carcinomas, endometrial carcinomas, liver metastases, penile cancer, tongue cancer, gall bladder cancer, leukaemia, plasmocytomas, uterine cancer, lid tumour and prostate cancer. Further examples of diseases that may be treated according to the invention include, but are not limited to, influenza, malaria, SARS, yellow fever, AIDS, Lyme borreliosis, Leishmaniasis, anthrax, meningitis, viral infectious diseases, AIDS, Condyloma acuminata, hollow warts, Dengue fever, three-day fever, Ebola virus, cold, early summer meningoencephalitis (FSME), flu, shingles, hepatitis, herpes simplex type I, herpes simplex type II, Herpes zoster, influenza, Japanese encephalitis, Lassa fever, Marburg virus, measles, foot-and-mouth disease, mononucleosis, mumps, Norwalk virus infection, Pfeiffer's glandular fever, smallpox, polio, pseudo-croup, German measles, rabies, warts, West Nile fever, chickenpox, cytomegalic virus (CMV), bacterial infectious diseases, miscarriage, prostate inflammation, anthrax, appendicitis, borreliosis, botulism, Camphylobacter, Chlamydia trachomatis, inflammation of the urethra, conjunctivitis, cholera, diphtheria, donavanosis, epiglottitis, typhus fever, gas gangrene, gonorrhoea, rabbit fever, Heliobacter pylori, whooping cough, climatic bubo, osteomyelitis, Legionnaire's disease, leprosy, listeriosis, pneumonia, meningitis, bacterial meningitis, anthrax, otitis media, Mycoplasma hominis, neonatal sepsis, Chorioamnionitis, noma, paratyphus, plague, Reiter's syndrome, Rocky Mountain spotted fever, Salmonella paratyphus, Salmonella typhus, scarlet fever, syphilis, tetanus, tripper, tsutsugamushi disease, tuberculosis, typhus, vaginitis, colpitis, soft chancre, parasitic infectious diseases, protozoal infectious diseases, fungal infectious diseases, amoebiasis, bilharziosis, Chagas disease, athlete's foot, yeast fungus spots, scabies, malaria, onchocercosis (river blindness), toxoplasmosis, trichomoniasis, trypanosomiasis (sleeping sickness), visceral Leishmaniosis, nappy dermatitis, schistosomiasis, fish poisoning (Ciguatera), candidosis, cutaneous Leishmaniosis, lambliasis (giardiasis), sleeping sickness, infectious diseases caused by Echinococcus, infectious diseases caused by fish tapeworm, infectious diseases caused by fox tapeworm, infectious diseases caused by canine tapeworm, infectious diseases caused by lice, infectious diseases caused by bovine tapeworm, infectious diseases caused by porcine tapeworm and infectious diseases caused by miniature tapeworm.

The present invention also contemplates kits comprising an immune-stimulating adjuvant of the invention. Such kits may further comprise technical instructions with information on the administration and dosage of the immune-stimulating adjuvant. Kits of the invention also include kits comprising the pharmaceutical compositions of the invention. Such kits may further comprise technical instructions with information on the administration and dosage of the pharmaceutical composition.

### BRIEF DESCRIPTION OF THE FIGURES

FIG. 1 shows various possibilities according to the invention for the terminal modification of nucleic acids with lipids. There are shown in particular the lipid-modified linkers or bifunctional peptides which can be used for coupling or synthesis with nucleic acid sequences (ODN sequence for short).

FIG. 2 describes by way of example a synthesis route for (trifunctional) lipid-modified linkers, with which, for example, a tocopherol modification can be introduced at the 3′ end of a nucleic acid. Such compounds shown by way of example represent an intermediate in the preparation of the 5′- or 3′-lipid-modified nucleic acids according to the invention and of the adjuvants according to the invention.

FIG. 3 shows by way of example a bifunctional lipid with a succinyl anchor, which permits a 3′-modification of a nucleic acid with a bifunctional lipid, for example with PEG.

FIG. 4 shows diagrammatically the coupling of lipid-modified amidites to the 5′ end of nucleic acids.

FIGS. 5A and 5B describe the stimulation of human PBMCs with immune-stimulating adjuvants according to the invention and with various RNA oligonucleotides. 5A) In particular in the case of the release of cytokines (IL-6), it is to be observed that the immune-stimulating adjuvants according to the invention without the addition of protamine exhibit a more than 5-fold increase in cytokine release (IL-6) as compared with the medium and, on addition of protamine, a slightly improved release of IL-6 as compared with β-galactosidase and RNA oligo 40 alone (SEQ ID NO: 40). 5B) When determining the TNF-α release, a marked stimulation of the immune system can be detected, which is at least equivalent to that of β-galactosidase or RNA.

FIG. 6 shows the release of TNF-α by human PBMC cells after stimulation with RNA oligonucleotides used according to the invention and with immune-stimulating adjuvants according to the invention. FIG. 6 shows in particular that immune-stimulating adjuvants according to the invention in the form of a lipid-modified nucleic acid, containing, for example, one of the sequences SEQ ID NO: 40, 41 or 42, exhibits a markedly improved release of TNF-α and accordingly markedly improved immune stimulation as compared with, for example, an unmodified RNA oligonucleotide having the sequence according to SEQ ID NO: 40 (RNA oligo 40). The best results, with a more than 10-fold increase in immune stimulation as compared with the unmodified RNA oligonucleotide, were achieved with a tocopherol-modified sequence according to SEQ ID NO: 42 (RNA oligo Toc CV2).

### DETAILED DESCRIPTION OF THE PREFERRED EMBODIMENTS

An “immune-stimulating” adjuvant according to the present invention is preferably capable of triggering an immune reaction. An immune reaction can generally be brought about in various ways. A substantial factor for a suitable immune response is the stimulation of different T-cell sub-populations. T-lymphocytes are typically divided into two sub-populations, the T-helper 1 (Th1) cells and the T-helper 2 (Th2) cells, with which the immune system is capable of destroying intracellular (Th1) and extracellular (Th2) pathogens (e.g. antigens). The two Th cell populations differ in the pattern of the effector proteins (cytokines) produced by them. Thus, Th1 cells assist the cellular immune response by activation of macrophages and cytotoxic T-cells. Th2 cells, on the other hand, promote the humoral immune response by stimulation of the B-cells for conversion into plasma cells and by formation of antibodies (e.g. against antigens). The Th1/Th2 ratio is therefore of great importance in the immune response. In connection with the present invention, the Th1/Th2 ratio of the immune response is preferably shifted by the adjuvant according to the invention in the direction towards the cellular response (Th1 response) and a cellular immune response is thereby induced.

The nucleic acid used according to the invention for the lipid-modified nucleic acid (adjuvant) can be a RNA or DNA (for example a cDNA), a RNA or DNA oligonucleotide, a RNA or DNA homopolymer, a CpG nucleic acid, etc. It can be single-stranded or double-stranded, in the form of a homo- or hetero-duplex, and linear or circular. The nucleic acid used according to the invention for the lipid-modified nucleic acid (adjuvant) is particularly preferably in the form of single-stranded RNA.

The lipid-modified nucleic acid is typically relatively short nucleic acid molecules consisting of, for example, from approximately 2 to approximately 1000 nucleotides, preferably of approximately from 5 to 200, from 6 to approximately 200 nucleotides, and particularly preferably of from 6 to approximately 40 or from 6 to approximately 31 nucleotides. In this connection, nucleotides are preferably any naturally occurring nucleotides and their analogues, such as ribonucleotides and/or deoxyribonucleotides, and include, without implying any limitation, for example, purines (adenine (A), guanine (G)) or pyrimidines (thymine (T), cytosine (C), uracil (U)) and also analogues or derivatives of purines and pyrimidines, such as, for example, 1-methyl-adenine, 2-methyl-adenine, 2-methylthio-N6-isopentenyl-adenine, N6-methyl-adenine, N6-isopentenyl-adenine, 2-thio-cytosine, 3-methyl-cytosine, 4-acetyl-cytosine, 5-methyl-cytosine, 2,6-diaminopurine, 1-methyl-guanine, 2-methyl-guanine, 2,2-dimethyl-guanine, 7-methyl-guanine, inosine, 1-methyl-inosine, dihydro-uracil, 2-thio-uracil, 4-thio-uracil, 5-carboxymethylaminomethyl-2-thio-uracil, 5-(carboxyhydroxylmethyl)-uracil, 5-fluoro-uracil, 5-bromo-uracil, 5-carboxymethylaminomethyl-uracil, 5-methyl-2-thio-uracil, 5-methyl-uracil, N-uracil-5-oxyacetic acid methyl ester, 5-methylaminomethyl-uracil, 5-methoxyaminomethyl-2-thio-uracil, 5′-methoxycarbonylmethyl-uracil, 5-methoxy-uracil, uracil-5-oxyacetic acid methyl ester, uracil-5-oxyacetic acid (v), pseudouracil, 1-methyl-pseudouracil, queosine, β-D-mannosyl-queosine, wybutoxosine, and also phosphoramidates, phosphorothioates, peptide nucleotides, methylphosphonates, 7-deazaguanosine 5-methylcytosine and inosine. The preparation of such analogues is known to the person skilled in the art, for example from U.S. Pat. No. 4,373,071, U.S. Pat. No. 4,401,796, U.S. Pat. No. 4,415,732, U.S. Pat. No. 4,458,066, U.S. Pat. No. 4,500,707, U.S. Pat. No. 4,668,777, U.S. Pat. No. 4,973,679, U.S. Pat. No. 5,047,524, U.S. Pat. No. 5,132,418, U.S. Pat. No. 5,153,319, U.S. Pat. Nos. 5,262,530 and 5,700,642, the disclosures of which are incorporated by reference herein in their entirety.

The lipid-modified nucleic acid can include any naturally occurring nucleic acid sequence, the complement thereof or a fragment thereof. In this context, a fragment of such a nucleic acid sequence preferably has a length of preferably approximately from 5 to 200, from 6 to approximately 200 nucleotides, and particularly preferably from 6 to approximately 40 or from 6 to approximately 31 nucleotides. The lipid-modified nucleic acid can also be partially or wholly of synthetic nature.

According to a first preferred embodiment there is used in the lipid-modified nucleic acid CpG nucleic acid, in particular CpG-RNA or CpG-DNA. A CpG-RNA or CpG-DNA used according to the invention can be a single-stranded CpG-DNA (ss CpG-DNA), a double-stranded CpG-DNA (dsDNA), a single-stranded CpG-RNA (ss CpG-RNA) or a double-stranded CpG-RNA (ds CpG-RNA). The CpG nucleic acid used according to the invention is preferably in the form of CpG-RNA, more preferably in the form of single-stranded CpG-RNA (ss CpG-RNA). Also preferably, such CpG nucleic acids have a length as described above.

The CpG nucleic acid used according to the invention preferably contains at least one or more (mitogenic) cytosine/guanine dinucleotide sequence(s) (CpG motif(s)), which are represented by the generic formulae 5′-X₁X₂CGX₃X₄-3′ (“hexamer”, SEQ ID NO: 1) or 5′-X₁X₂X₃CGX₄X₅X₆-3′ (“octamer”, SEQ ID NO: 2). According to a first preferred alternative, at least one CpG motif contained in these hexamer or octamer sequences, that is to say the C (cytosine) and the G (guanine) of the CpG motif, is unmethylated. All further cytosines or guanines optionally contained in the hexamer or octamer sequences can be either methylated or unmethylated. According to a further preferred alternative, however, the C (cytosine) and the G (guanine) of the CpG motif can also be present in methylated form. Within the context of the above-mentioned hexamer or octamer sequences, X₁, X₂, X₃, X₄, X₅ and X₆ preferably represent nucleotides which can be selected independently of one another or together from all naturally occurring nucleotides and their analogues, as described generally above for nucleic acids used herein.

According to a preferred embodiment, the CpG nucleic acid used according to the invention contains as CpG motif at least one or more octamers selected from the group consisting of: GACGTTCC (SEQ ID NO: 3); GACGCTCC (SEQ ID NO: 4); GACGTCCC (SEQ ID NO: 5); GACGCCCC (SEQ ID NO: 6); AGCGTTCC (SEQ ID NO: 7); AGCGCTCC (SEQ ID NO: 8); AGCGTCCC (SEQ ID NO: 9); AGCGCCCC (SEQ ID NO: 10); AACGTTCC (SEQ ID NO: 11); AACGCTCC (SEQ ID NO: 12); AACGTCCC (SEQ ID NO: 13); AACGCCCC (SEQ ID NO: 14); GGCGTTCC (SEQ ID NO: 15); GGCGCTCC (SEQ ID NO: 16); GGCGTCCC (SEQ ID NO: 17); GGCGCCCC (SEQ ID NO: 18); GACGTTCG (SEQ ID NO: 19); GACGCTCG (SEQ ID NO: 20); GACGTCCG (SEQ ID NO: 21); GACGCCCG (SEQ ID NO: 22); AGCGTTCG (SEQ ID NO: 23); AGCGCTCG (SEQ ID NO: 24); AGCGTCCG (SEQ ID NO: 25); AGCGCCCG (SEQ ID NO: 26); AACGTTCG (SEQ ID NO: 27); AACGCTCG (SEQ ID NO: 28); AACGTCCG (SEQ ID NO: 29); AACGCCCG (SEQ ID NO: 30); GGCGTTCG (SEQ ID NO: 31); GGCGCTCG (SEQ ID NO: 32); GGCGTCCG (SEQ ID NO: 33); GGCGCCCG (SEQ ID NO: 34). Most preferably, the CpG nucleic acid used according to the invention contains as CpG motif at least one or more octamers selected from the group consisting of: GACGTTCC (SEQ ID NO: 3), AACGTTCC (SEQ ID NO: 11), GACGTTCG (SEQ ID NO: 19) and AACGTTCG (SEQ ID NO: 23). Also included are those sequences that are at least 60%, more preferably 70 or 80% and most preferably 90 or 95% identical with one of the preceding sequences. In order to determine the percentage identity of two nucleic acid sequences with one another, the sequences can be aligned and subsequently compared with one another. To this end, gaps, for example, can be introduced into the sequence of the first nucleic acid sequence, and the nucleotides at the corresponding position of the second nucleic acid sequence can be compared. When a position in the first nucleic acid sequence is occupied with the same nucleotide as in a position in the second sequence, then the two sequences are identical at that position. Determination of the percentage identity of two sequences can be carried out by means of a mathematical algorithm. A preferred, but non-limiting example of a mathematical algorithm which can be used for comparing two sequences is the algorithm of Karlin et al. (1993), PNAS USA, 90:5873-5877. Such an algorithm is integrated into the NBLAST program, with which sequences having a desired identity with the sequences of the present invention can be identified. In order to obtain a gapped alignment as described above, the “Gapped BLAST” program can be used, as described in Altschul et al. (1997), Nucleic Acids Res, 25:3389-3402.

The mitogenic CpG motif contained in the CpG nucleic acid used according to the invention, more preferably a hexamer or octamer according to one of sequences SEQ ID NO: 1 to 34, preferably occurs at least once in the CpG nucleic acid. Particularly preferably, no GCG trinucleotide occurs at the 5′ or 3′ end or close to the 5′ or 3′ end of the CpG nucleic acid used, for example in a range of from 1 to 3 or from 1 to 6 nucleotides. The hexamer or octamer sequences according to one of sequences SEQ ID NO: 1 to 34 can occur at least once in the CpG nucleic acid used according to the invention, i.e. at least one hexamer and/or octamer sequence according to one of sequences SEQ ID NO: 1 to 34 is present. Alternatively, the hexamer or octamer sequences according to one of sequences SEQ ID NO: 1 to 34 can occur in the CpG nucleic acid used according to the invention in the form of a multimer, for example as a sequence of 2 to 5, 5 to 10, 10 to 15, 15 to 20, 20 to 30, 30 to 40, 40 to 50 or 50 to 100 of the sequences according to SEQ ID NO: 1 to 34, wherein the hexamer and octamer sequences can be combined with one another as desired. The individual hexamer or octamer sequences according to SEQ ID NO: 1 to 34 can be separated from one another by 1 to 30, preferably 1 to 20, more preferably 1 to 10 of the above-mentioned nucleotides, or alternatively they can follow one another directly without intermediate nucleotides.

The CpG nucleic acid used according to the invention can further be present in the form of a “stabilised oligonucleotide”, i.e. in the form of an oligoribonucleotide or oligodeoxyribonucleotide that is resistant to in vivo degradation (e.g. by an exo- or endo-nuclease). Such stabilisation can be effected, for example, by a modified phosphate backbone of the CpG nucleic acid used according to the invention. Nucleotides that are preferably used in this connection contain a phosphorothioate-modified phosphate backbone, preferably at least one of the phosphate oxygens contained in the phosphate backbone being replaced by a sulfur atom. Other stabilised oligonucleotides include, for example: non-ionic analogues, such as, for example, alkyl and aryl phosphonates, in which the charged phosphonate oxygen is replaced by an alkyl or aryl group, or phosphodiesters and alkylphosphotriesters, in which the charged oxygen radical is present in alkylated form.

According to a further preferred embodiment, the nucleic acid used for the lipid-modified nucleic acid is present in the form of a RNA or DNA homopolymer, more preferably in the form of a RNA homopolymer. Such a DNA or RNA homopolymer typically includes single-stranded or double-stranded, preferably single-stranded, polynucleotides such as, for example, polyinosinic acid (I), polyadenic acid (A), polyuridic acid (U), polyxanthinic acid (X) or polyguanylic acid (G). The RNA or DNA homopolymers used according to the invention can occur in the form of single-stranded RNA or DNA homopolymers. Such RNA or DNA homopolymers are well known in the prior art and typically do not have a uniform molecular weight. Molecular weights of double-stranded complexes of copolymers have been determined, for example, in a range of approximately from 1×10⁵ to 1.5×10⁶.

A first preferred alternative of the RNA or DNA homopolymers includes single-stranded RNA or DNA homopolymers. Such single-stranded RNA or DNA homopolymers typically contain a ribonucleotide or deoxyribonucleotide as defined above in n-fold repetition, n preferably being equal to the length of the above-described nucleic acids used according to the invention and being in a range of from 2 to approximately 1000, preferably from 5 to 200, more preferably from 6 to approximately 200, and most preferably from 6 to approximately 40 or from 6 to approximately 31. Particularly preferred single-stranded RNA or DNA homopolymers include, without implying any limitation, the following sequences: 5′-AAAAAAAAAAAAAAAAAAAAAA-3′ (SEQ ID NO: 35), 5′-UUUUUUUUUUUUUUUUUUUUUU-3′ (SEQ ID NO: 36), 5′-GGGGGGGGGGGGGGGGGGGGGG-3′ (SEQ ID NO: 37), 5′-CCCCCCCCCCCCCCCCCCCCCC-3′ (SEQ ID NO: 38) and 5′-TTTTTTTTTTTTTTTTTTTTTT-3′ (SEQ ID NO: 39). Also included are those sequences that are at least 60%, more preferably at least 70 or 80% and most preferably at least 90 or 95% identical with one of the preceding sequences.

Chemically altered polynucleotides represent a second preferred alternative of the DNA or RNA homopolymers. Chemically altered polynucleotides within the scope of the present invention can be DNA or RNA polymers as described above that contain in their sequence at least one nucleotide, for example an analogue or derivative of purines (adenine (A), guanine (G)) or pyrimidines (thymine (T), cytosine (C), uracil (U)), as described above. More preferably, such chemically altered polynucleotides have a content of analogues and derivatives of from 1 to 100%, for example 1 to 20, 10 to 30, 20 to 40, 30 to 50, 40 to 60, 50 to 70, 60 to 80, 70 to 90 or 80 to 100%. Such chemically altered polynucleotides can likewise be prepared by processes known in the prior art (see processes for the preparation of complexes of homopolymers). Examples of chemically altered polynucleotides include, without implying any limitation, compounds such as, for example, poly-N₁-methyladenylate, poly-“6-methyladenylate”, poly-N7-methylinosate, poly-N7-methylguanylate, poly-5-methyluridylate, poly-5-fluorouridylate, poly-5-bromouridylate, poly-5-bromocytidylate and poly-5-iodocytidylate, etc.

According to a sixth alternative, there can be used as RNA or DNA homopolymers also combinations of the above-described RNA or DNA homopolymers. Such combinations preferably include nucleic acid sequences that contain at least two of the above-mentioned alternatives of the RNA or DNA homopolymers described herein or multimers thereof, for example a sequence of 2 to 5, 5 to 10, 10 to 15, 15 to 20, 20 to 30, to 40, 40 to 50 or 50 to 100 of one or more of the above-described RNA or DNA homopolymers, particularly preferably according to one of SEQ ID NO: 35 to 39. The individual RNA or DNA homopolymers can be separated from one another by 1 to 30, preferably 1 to 20, more preferably 1 to 10, of the above-described nucleotides, or alternatively they can follow one another directly without intermediate nucleotides.

According to a further preferred embodiment, there can be used for the lipid-modified nucleic acid those nucleic acids that cannot be assigned to any of the above-mentioned nucleic acid classes and are already known as immunogenic in the prior art, or that are not yet known in the prior art but have immunogenic properties. Nucleic acids according to this alternative are preferably in the form of RNA or DNA, more preferably in the form of RNA. Also preferably, these nucleic acids have a length as described above and contain nucleotides, for example ribonucleotides or deoxyribonucleotides, as disclosed hereinbefore. Such nucleic acids can code for antigens, for example. Alternatively, such nucleic acids can code for epitopes (of proteins). Such nucleic acids then preferably have an ATG as start signal, which marks the start of the translation of the coded RNA. According to a particularly preferred embodiment, the nucleic acid sequence of the lipid-modified nucleic acid, for example, contains at least one sequence according to one of SEQ ID NOs: 40-67, as listed hereinbelow: 5′-GCCCGUCUGUUGUGUGACUC-3′ (SEQ ID NO: 40, also referred to as RNA 40), 5′-GGUAAGUGUAAGGUGUAAGG-3′ (SEQ ID NO: 41, also referred to as RNA CV1), 5′-AAUGGAUAUGGAAUAUGGAA-3′ (SEQ ID NO: 42, also referred to as RNA CV2), 5′-UCCAUGACGUUCCUGACGUU-3′ (SEQ ID NO: 43), 5′-UCCAGGACUUCUCUCAGGUU-3′ (SEQ ID NO: 44), 5′-UCCAUGACGUUCCUGAUGCU-3′ (SEQ ID NO: 45), 5′-GCCCGUCUGUUGUGUGACUC-3′ (SEQ ID NO: 46), 5′-GGUAAGUGUAAGGUGUAAGG-3′ (SEQ ID NO: 47), 5′-AAUGGAUAUGGAAUAUGGAA-3′ (SEQ ID NO: 48), 5′-CUCUGGAGGAAAAGAAAGUTT-3′ (SEQ ID NO: 49), 5′-CAAUGCAACUCGCUUCUCGTT-3′ (SEQ ID NO: 50), 5′-AGCUUAACCUGUCCUUCAA-3′ (SEQ ID NO: 51), 5′-AAAAAAAACUGUCCUUCAA-3′ (SEQ ID NO: 52), 5′-AAAAAAAAAUGUCCUUCAA-3′ (SEQ ID NO: 53), 5′-AAAAAAAAAAGUCCUUCAA-3′ (SEQ ID NO: 54), 5′-AAAAAAAAAAAUCCUUCAA-3′ (SEQ ID NO: 55), 5′-AAAAAAAAAAAACCUUCAA-3′ (SEQ ID NO: 56), 5′-AGCUUAACCUGUCCUUAAA-3′ (SEQ ID NO: 57), 5′-AGCUUAACCUGUCCUAAAA-3′ (SEQ ID NO: 58), 5′-AGCUUAACCUGUCCAAAAA-3′ (SEQ ID NO: 59), 5′-AGCUUAACCUGAAAAAAAA-3′ (SEQ ID NO: 60), 5′-UGUCCUUCAAUGUCCUUCAA-3′ (SEQ ID NO: 61) 5′-AGCUUAACCUGUCCUUCAU-3′ (SEQ ID NO: 62), 5′-AGCUUAACCUGUCCUUCUU-3′ (SEQ ID NO: 63), 5′-AGCUUAACCUGUCCUUCAACUACA-3′ (SEQ ID NO: 64), 5′-CAAAUUGAAGGACAGGUUAAGCU-3′ (SEQ ID NO: 65), 5′-UUAACCUGUCCUUCAA-3′ (SEQ ID NO: 66), 5′-AACCUGUCCUUCA-3′ (SEQ ID NO: 67). Also included are those sequences that are at least 60%, more preferably 70 or 80% and most preferably 90 to 95% identical with one of the preceding sequences.

According to another embodiment it is possible to use for the lipid-modified nucleic acid also those nucleic acids that represent a multimer of one or more of the above-described nucleic acids, for example a sequence of 2 to 5, 5 to 10, 10 to 15, 15 to 20, 20 to 30, 30 to 40, 40 to 50 or 50 to 100 of the above-described nucleic acids, particularly preferably according to SEQ ID NO: 1 to 67. The sequence of the nucleic acids can be chosen in this as desired. The individual nucleic acids/nucleic acid units of the multimer can be separated from one another by 1 to 30, preferably 1 to 20, more preferably 1 to 10 of the above-described nucleotides, or alternatively they can follow one another directly without intermediate nucleotides.

The nucleic acid used for the lipid-modified nucleic acid according to the invention can additionally contain, apart from the lipid modification, at least one chemical modification. According to the invention, preference is given especially to those chemical modifications that increase the immunogenity of the adjuvant according to the invention or do not interfere with the lipid modification of the nucleic acid used according to the invention. For example, if the lipid modification is present at the 3′ end of the nucleic acid used according to the invention, chemical modifications can typically be introduced at the 5′ end and/or within the sequence of the nucleic acid used according to the invention. If the lipid modification is present at the 5′ end of the nucleic acid used according to the invention, chemical modifications can typically be introduced at the 3′ end and/or within the sequence of the nucleic acid used according to the invention. If, on the other hand, the lipid modification is present at the 3′ end and at the 5′ end of the nucleic acid used according to the invention, the chemical modification is preferably introduced within the sequence of the nucleic acid used according to the invention.

The form of the chemical modification of the lipid-modified nucleic acid according to the invention is preferably such that the nucleic acid used therefor, preferably RNA, contains at least one analogue of naturally occurring nucleotides. Such analogues include the nucleotides described hereinbefore and their analogues. In addition, all the above-mentioned nucleotides and their analogues can be further chemically modified by, for example, acetylation, methylation, hydroxylation, etc. and used according to the invention.

The nucleic acid used according to the invention for the lipid-modified nucleic acid, or the lipid-modified nucleic acid itself, can further be stabilised. As mentioned above, any nucleic acid can in principle be used for the lipid-modified nucleic acid. From the point of view of safety, however, the use of RNA for such a nucleic acid is preferred. In particular, RNA does not involve the risk of being stably integrated into the genome of the transfected cell. In addition, RNA is degraded substantially more easily in vivo. Likewise, no anti-RNA antibodies have hitherto been detected, presumably owing to the relatively short half-life of RNA in the bloodstream as compared with DNA. In comparison with DNA, RNA is considerably less stable in solution, however, which is due substantially to RNA-degrading enzymes, so-called RNases (ribonucleases). Even the smallest ribonuclease contaminations are sufficient to degrade RNA completely in solution. Such RNase contaminations can generally be removed only by special treatment, in particular with diethyl pyrocarbonate (DEPC). The natural degradation of mRNA in the cytoplasm of cells is very finely regulated. A number of mechanisms are known in this connection in the prior art. Thus, the terminal structure is typically of critical importance for a RNA in vivo. At the 5′ end of naturally occurring RNAs there is usually a so-called “cap structure” (a modified guanosine nucleotide) and at the 3′ end a sequence of up to 200 adenosine nucleotides (the so-called poly-A tail).

The nucleic acid of the lipid-modified nucleic acid, if present in the form of RNA, can therefore be stabilised against degradation by RNases by the addition of a so-called “5′ cap” structure. Particular preference is given in this connection to a m7G(5′)ppp (5′(A,G(5′)ppp(5′)A or G(5′)ppp(5′)G as the 5′ cap structure. However, such a modification is introduced only if a modification, for example a lipid modification, has not already been introduced at the 5′ end of the nucleic acid used according to the invention.

Alternatively, the 3′ end of the nucleic acid of the lipid-modified nucleic acid, if present in the form of RNA, can be modified by a sequence of at least 50 adenosine nucleotides, preferably at least 70 adenosine nucleotides, more preferably at least 100 adenosine nucleotides, particularly preferably at least 200 adenosine nucleotides. Analogously, such a modification can be introduced only if a modification, for example a lipid modification, has not already been introduced at the 3′ end of the nucleic acid used according to the invention.

The lipid contained in the lipid-modified nucleic acid according to the invention is typically a lipid or a lipophilic radical that preferably is itself biologically active. Such lipids preferably include natural substances or compounds such as, for example, vitamins, e.g. α-tocopherol (vitamin E), including RRR-α-tocopherol (formerly D-α-tocopherol), L-α-tocopherol, the racemate D,L-α-tocopherol, vitamin E succinate (VES), or vitamin A and its derivatives, e.g. retinoic acid, retinol, vitamin D and its derivatives, e.g. vitamin D and also the ergosterol precursors thereof, vitamin E and its derivatives, vitamin K and its derivatives, e.g. vitamin K and related quinone or phytol compounds, or steroids, such as bile acids, for example cholic acid, deoxycholic acid, dehydrocholic acid, cortisone, digoxygenin, testosterone, cholesterol or thiocholesterol. Further lipids or lipophilic radicals within the scope of the present invention include, without implying any limitation, polyalkylene glycols (Oberhauser et al., Nucl. Acids Res., 1992, 20, 533), aliphatic groups such as, for example, C₁-C₂₀-alkanes, C₁-C₂₀-alkenes or C₁-C₂₀-alkanol compounds, etc., such as, for example, dodecanediol, hexadecanol or undecyl radicals (Saison-Behmoaras et al., EMBO J, 1991, 10, 111; Kabanov et al., FEBS Lett., 1990, 259, 327; Svinarchuk et al., Biochimie, 1993, 75, 49), phospholipids such as, for example, phosphatidylglycerol, diacylphosphatidylglycerol, phosphatidylcholine, dipalmitoylphosphatidylcholine, distearoylphosphatidylcholine, phosphatidylserine, phosphatidylethanolamine, di-hexadecyl-rac-glycerol, sphingolipids, cerebrosides, gangliosides, or triethylammonium 1,2-di-O-hexadecyl-rac-glycero-3-H-phosphonate (Manoharan et al., Tetrahedron Lett., 1995, 36, 3651; Shea et al., Nucl. Acids Res., 1990, 18, 3777), polyamines or polyalkylene glycols, such as, for example, polyethylene glycol (PEG) (Manoharan et al., Nucleosides & Nucleotides, 1995, 14, 969), hexaethylene glycol (HEG), palmitin or palmityl radicals (Mishra et al., Biochim. Biophys. Acta, 1995, 1264, 229), octadecylamines or hexylamino-carbonyl-oxycholesterol radicals (Crooke et al., J. Pharmacol. Exp. Ther., 1996, 277, 923), and also waxes, terpenes, alicyclic hydrocarbons, saturated and mono- or poly-unsaturated fatty acid radicals, etc.

Linking between the lipid and the nucleic acid used according to the invention can in principle take place at any nucleotide, at the base or the sugar radical of any nucleotide, at the 3′ and/or 5′ end, and/or at the phosphate backbone of the nucleic acid used according to the invention. Particular preference is given according to the invention to a terminal lipid modification of the nucleic acid used according to the invention at the 3′ and/or 5′ end thereof. A terminal modification has a number of advantages over modifications within the sequence. On the one hand, modifications within the sequence can influence the hybridisation behaviour, which may have an adverse effect in the case of sterically demanding radicals. On the other hand, in the case of the synthetic preparation of a lipid-modified nucleic acid according to the invention that is modified only terminally, the synthesis of the nucleic acid sequence can be carried out with commercially available monomers that are obtainable in large quantities, and synthesis protocols known in the prior art can be used.

According to a first preferred embodiment, linking between the nucleic acid used according to the invention and at least one lipid that is used is effected via a “linker” (covalently linked with the nucleic acid). Linkers within the scope of the present invention typically have at least two and optionally 3, 4, 5, 6, 7, 8, 9, 10, 10-20, 20-30 or more reactive groups, in each case selected from, for example, a hydroxy group, an amino group, an alkoxy group, etc. One reactive group preferably serves to bind the above-described nucleic acid used according to the invention, for example a RNA oligonucleotide. This reactive group can be present in protected form, for example as a DMT group (dimethoxytrityl chloride), as a Fmoc group, as a MMT (monomethoxytrityl) group, as a TFA (trifluoroacetic acid) group, etc. Furthermore, sulfur groups can be protected by disulfides, for example alkylthiols such as, for example, 3-thiopropanol, or by activated components such as 2-thiopyridine. One or more further reactive groups serve according to the invention for the covalent binding of one or more lipids. According to the first embodiment, therefore, a nucleic acid used according to the invention can bind via the covalently bound linker preferably at least one lipid, for example 1, 2, 3, 4, 5, 5-10, 10-20, 20-30 or more lipid(s), particularly preferably at least 3-8 or more lipid(s) per nucleic acid. The bound lipids can thereby be bound separately from one another at different positions of the nucleic acid, or they can be present in the form of a complex at one or more positions of the nucleic acid. An additional reactive group of the linker can be used for direct or indirect (cleavable) binding to a carrier material, for example a solid phase. Preferred linkers according to the present invention are, for example, glycol, glycerol and glycerol derivatives, 2-aminobutyl-1,3-propanediol and 2-aminobutyl-1,3-propanediol derivatives/scaffold, pyrrolidine linkers or pyrrolidine-containing organic molecules (in particular for a modification at the 3′ end), etc. Glycerol or glycerol derivatives (C₃ anchor) or a 2-aminobutyl-1,3-propanediol derivative/scaffold (C₇ anchor) are particularly preferably used according to the invention as linkers. A glycerol derivative (C₃ anchor) as linker is particularly preferred when the lipid modification can be introduced via an ether bond. If the lipid modification is to be introduced via an amide or a urethane bond, for example, a 2-aminobutyl-1,3-propanediol scaffold (C₇ anchor), for example, is preferred. In this connection, the nature of the bond formed between the linker and the nucleic acid used according to the invention is preferably such that it is compatible with the conditions and chemicals of amidite chemistry, that is to say it is preferably neither acid- nor base-labile. Preference is given in particular to bonds that are readily obtainable synthetically and are not hydrolysed by the ammoniacal cleavage procedure of a nucleic acid synthesis process. Suitable bonds are in principle all correspondingly suitable bonds, preferably ester bonds, amide bonds, urethane and ether bonds. In addition to the good accessibility of the starting materials (few synthesis steps), particular preference is given to the ether bond owing to its relatively high biological stability towards enzymatic hydrolysis.

According to a second preferred embodiment, the (at least one) nucleic acid used according to the invention is linked directly with at least one (bifunctional) lipid as described above, i.e. without the use of a linker as described above. In this case, the (bifunctional) lipid used according to the invention preferably contains at least two reactive groups or optionally 3, 4, 5, 6, 7, 8, 9, 10 or more reactive groups, a first reactive group serving to bind the lipid directly or indirectly to a carrier material described herein and at least one further reactive group serving to bind a nucleic acid used according to the invention. According to the second embodiment, a nucleic acid used according to the invention can therefore preferably bind at least one lipid (directly without a linker), for example 1, 2, 3, 4, 5, 5-10, 10-20, 20-30 or more lipid(s), particularly preferably at least 3-8 or more lipid(s) per nucleic acid. The bound lipids can be bound separately from one another at different positions of the nucleic acid, or they can be present in the form of a complex at one or more positions of the nucleic acid. Alternatively, at least one nucleic acid, for example optionally 3, 4, 5, 6, 7, 8, 9, 10, 10-20, 20-30 or more nucleic acids, can be bound according to the second embodiment to a lipid as described above via its reactive groups. Lipids that can be used for this second embodiment particularly preferably include those (bifunctional) lipids that permit coupling (preferably at their termini or optionally intramolecularly), such as, for example, polyethylene glycol (PEG) and derivatives thereof, hexaethylene glycol (HEG) and derivatives thereof, alkanediols, aminoalkane, thioalkanols, etc. The nature of the bond between a (bifunctional) lipid and a nucleic acid, as described above, used according to the invention is preferably as described for the first preferred embodiment.

According to a third embodiment, linking between the nucleic acid used according to the invention and at least one lipid as described above can take place via both of the above-mentioned embodiments simultaneously. For example: the nucleic acid can be linked at one position of the nucleic acid with at least one lipid via a linker (analogously to the first embodiment) and at a different position of the nucleic acid directly with at least one lipid without the use of a linker (analogously to the second embodiment). For example, at the 3′ end of a nucleic acid used according to the invention, at least one lipid as described above can be covalently linked with the nucleic acid via a linker, and at the 5′ end of the nucleic acid, a lipid as described above can be covalently linked with the nucleic acid without a linker. Alternatively, at the 5′ end of a nucleic acid used according to the invention, at least one lipid as described above can be covalently linked with the nucleic acid via a linker, and at the 3′ end of the nucleic acid, a lipid as described above can be covalently linked with the nucleic acid without a linker. Likewise, covalent linking can take place not only at the termini of the nucleic acid but also intramolecularly, as described above, for example at the 3′ end and intramolecularly, at the 5′ end and intramolecularly, at the 3′ and 5′ end and intramolecularly, only intramolecularly, etc.

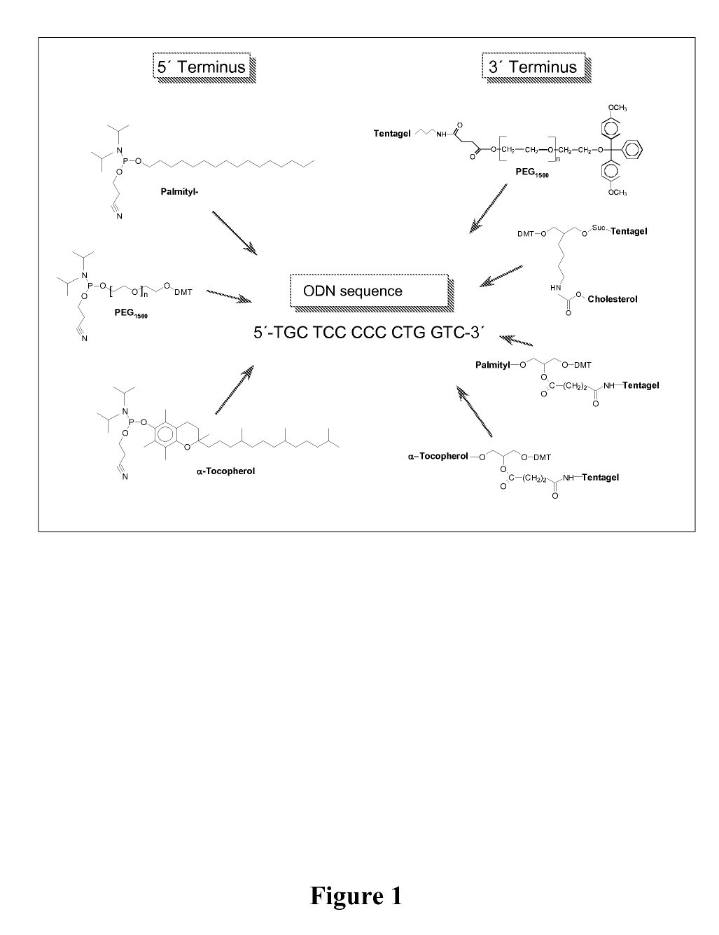

The lipid-modified nucleic acid(s) used as the adjuvant according to the invention can preferably be obtained by various processes. The lipid modification can in principle—as defined above—be introduced at any position of the nucleic acid sequence used, for example at the 3′ and/or 5′ ends or at the phosphate backbone of the nucleic acid sequence used and/or at any base or at the sugar of any nucleotide of the nucleic acid sequence used. According to the invention, preference is given to terminal lipid modifications at the 3′ and/or 5′ ends of the nucleic acids used. By means of such a terminal chemical modification it is possible according to the invention to obtain a large number of differently derivatised nucleic acids. Examples of variants included in the invention are shown in FIG. 1. The process for preparing such lipid-modified nucleic acids is preferably chosen in dependence on the position of the lipid modification.

If, for example, the lipid modification takes place at the 3′ end of the nucleic acid, then the lipid modification is typically carried out either before or after the preparation of the nucleic acid used according to the invention. The preparation of the nucleic acid used according to the invention can be carried out by direct synthesis of the nucleic acid or by addition of a ready synthesised (e.g. commercially available) nucleic acid or a nucleic acid isolated from samples.

According to a first alternative, the nucleic acid of a 3′-lipid-modified nucleic acid according to the invention is synthesised directly before introduction of the lipid, typically by means of processes known in the prior art for the synthesis of nucleic acids. To this end, a starting nucleoside is preferably bound to a solid phase, for example via a coupling molecule, e.g. a succinyl radical, and the nucleic acid is synthesised, for example by the process of amidite chemistry. A linker as described hereinbefore is then covalently bonded, preferably via a first reactive group of the linker, to the 3′ end of the nucleic acid. A lipid as described hereinbefore can then be covalently linked with the linker via a second reactive group of the linker. Alternatively, the linker can be covalently linked with the lipid before it is bound to the 3′ end of the nucleic acid. In this case, only the binding of a first reactive group of the linker with the 3′ end of the nucleic acid is necessary. After synthesis of the oligonucleotide, or after binding of the lipid, the nucleic acid can be separated from the solid phase and deprotected. If the synthesis has been carried out in solution, a washing and purification step for removing unreacted reactants as well as solvents and undesirable secondary products can be carried out after the synthesis of the lipid-modified nucleic acid (and optionally before separation from the carrier material).

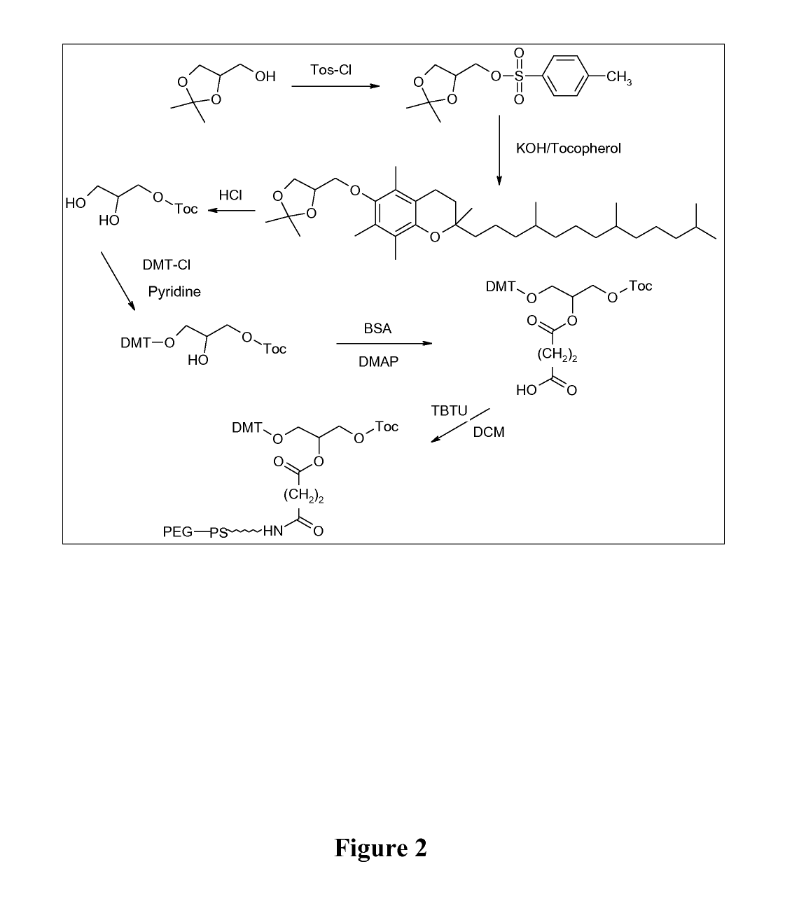

According to a further alternative, the nucleic acid of a 3′-lipid-modified nucleic acid according to the invention is synthesised after introduction of the lipid on a reactive group of the linker or is bound as a (commercially available) ready synthesised nucleic acid or a nucleic acid that has been isolated from samples to the reactive group of the linker (see e.g. FIG. 2). To this end, for example, a first reactive group of a linker as described above can be reacted with a lipid as described hereinbefore. Then, preferably in a second step, a second reactive group of the linker is provided with an acid-stable protecting group, e.g. DMT, Fmoc, etc., in order to permit subsequent binding of the nucleic acid to that reactive group. The linker can then be bound directly or indirectly to a solid phase via a third reactive group of the linker. Indirect binding is possible, for example, via a (coupling) molecule, which can be bound both covalently to the linker and to the solid phase. Such a (coupling) molecule is, for example, a succinyl radical, etc., as described hereinbelow. Removal of the protecting group at the third reactive group of the linker and the binding or synthesis of a nucleic acid at the reactive group that is now accessible then usually take place. Finally, the lipid-modified nucleic acid is typically removed from the carrier material (and the protective groups on the nucleic acid are optionally removed). However, a further lipid can optionally also be coupled to the 3′ end of the coupled nucleic acid, preferably according to one of the steps described hereinbefore.

According to a variant of this above-mentioned alternative, a linker as described above can be bound directly or indirectly to a solid phase via a first reactive group. An acid-stable protecting group is then first bound to a second reactive group of the linker. After binding of the protecting group to the second reactive group, a lipid as described above can first be bound to a third reactive group of the linker. Then there are likewise preferably carried out the removal of the protecting group at the third reactive group of the linker, the binding or synthesis of a nucleic acid at the reactive group that is now accessible, and the removal of the lipid-modified nucleic acid from the carrier material (and optionally the removal of the protecting groups at the nucleic acid).

According to a particularly preferred embodiment of the 3′-lipid modification, a lipid-modified nucleic acid can be synthesised via a linker having three reactive groups (a trifunctional anchor compound) based on a glycerol fundamental substance (C₃ anchor) and having a monofunctional lipid, such as, for example, a palmityl radical, cholesterol or tocopherol. As starting material for the synthesis of the linker there can be used, for example, α,β-isopropylidene-glycerol (a glycerol containing a ketal protecting group), which is preferably first converted into the alcoholate with sodium hydride and is reacted with hexadecyl bromide and a lipid in a Williamson synthesis to form the corresponding ether. Alternatively, the ether bond can be linked in the first step by a different method, for example by formation of a tosylate of α,β-isopropylidene-glycerol, and reaction of the tosylate with the reactive group of a lipid, for example an acidic proton, to form the corresponding ether. In a second stage, the ketal protecting group can be removed with an acid, for example acetic acid, dilute hydrochloric acid, etc., and then the primary hydroxy group of the diol can be protected selectively by dimethoxytrityl chloride (DMT-Cl). In the last stage, the reaction of the product obtained in the preceding step with succinic anhydride is preferably carried out to form the succinate with DMAP as catalyst. Such a linker is particularly suitable, for example, for the binding of palmityl radicals or tocopherol as lipid (see e.g. FIG. 2).

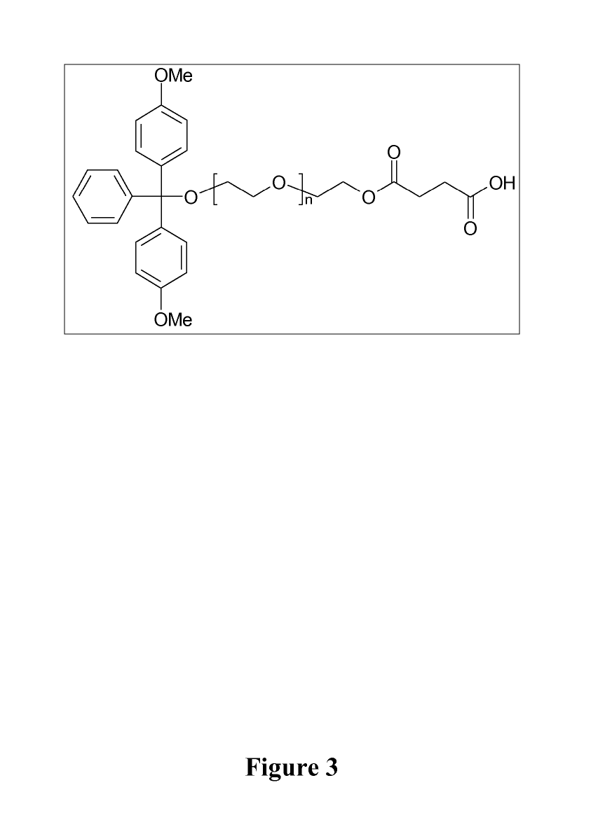

According to another alternative of the 3′-lipid modification, a lipid-modified nucleic acid can by the use of a (bifunctional) lipid, such as, for example, polyethylene glycol (PEG) or hexaethylene glycol (HEG), without using a linker as described above. Such bifunctional lipids typically have two functional groups as described above, wherein one end of the bifunctional lipid can preferably be bound to the carrier material via a (coupling) molecule, for example a base-labile succinyl anchor, etc., as described herein, and the nucleic acid can be synthesised at the other end of the bifunctional lipid (E. Bayer, M. Maier, K. Bleicher, H.-J. Gaus Z. Naturforsch. 50b (1995) 671). By the omission of the third functionalisation or of a linker as used hereinbefore, the synthesis of such a lipid-modified nucleic acid according to the invention is simplified (see e.g. FIG. 3). For the preparation, the bifunctional lipid used according to the invention, for example polyethylene glycol, is typically first monosubstituted with a protecting group, for example DMT. In a second stage, esterification of the lipid protected at a reactive group is usually carried out with succinic anhydride, with DMAP catalysis, to form the succinate. Thereafter, in a third stage, the bifunctional lipid can be coupled to a carrier material and deprotected, following which the synthesis of the nucleic acid takes place in a fourth stage in accordance with a process as described hereinbefore. Deprotection of the nucleic acid and separation of the lipid-modified nucleic acid from the carrier material are then optionally carried out.

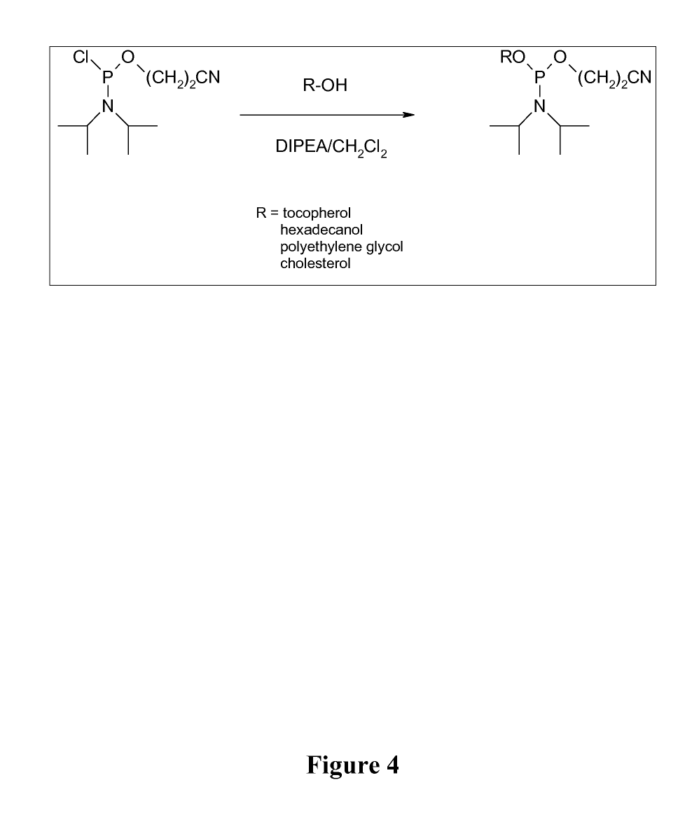

According to another preferred embodiment, the lipid modification takes place at the 5′ end of the nucleic acid. The lipid modification is thereby typically carried out either after the preparation or after the synthesis of the nucleic acid used according to the invention. The preparation of the nucleic acid according to the invention can be carried out—as defined above—via a direct synthesis of the nucleic acid or by addition of a ready synthesised nucleic acid or a nucleic acid isolated from samples, i.e. a commercially available nucleic acid. A synthesis of the nucleic acid takes place preferably analogously to the method described above, according to processes of nucleic acid synthesis known in the prior art, more preferably according to the phosphoramidite process (see e.g. FIG. 4).

According to a particularly preferred embodiment, the lipid modification takes place at the 5′ end of the nucleic acid used according to the invention by specially modified phosphoramidites following a phosphoramidite process for the synthesis of the nucleic acid. Such amidites, which are obtainable relatively simply by synthesis, are conventionally coupled as the last monomer to a commercially available or to a ready synthesised nucleic acid. These reactions are distinguished by a relatively rapid reaction kinetics and very high coupling yields. The synthesis of the modified amidites preferably takes place by reaction of a phosphoramidite, for example β-cyanoethyl-monochlorophosphoramidite (phosphorous acid mono-(2-cyanoethyl ester)-diisopropyl-amide chloride) with an alcohol, dissolved in a suitable solvent, for example in absolute dichloromethane, of a lipid as defined above, for example a lipid alcohol of tocopherol, cholesterol, hexadecanol, DMT-PEG, etc. Likewise preferably, DIPEA is added to the reaction solution as acid acceptor.

These phosphoramidites used for the synthesis of the 5′-lipid-modified nucleic acids according to the invention are relatively resistant to hydrolysis and can (prior to the synthesis) be purified chromatographically by means of silica gel. To this end, a small amount of a weak base, such as, for example, triethylamine, is typically added to the eluent in order to avoid decomposition of the amidite. It is important that this base is removed completely from the product again, in order to avoid poor coupling yields. This can be carried out, for example, by simple drying in vacuo, but preferably by purification of the phosphoramidites by precipitation thereof from tert-butyl methyl ether using pentane. If the lipid-modified amidites used have a very high viscosity, for example are present in the form of a viscous oil, (rapid) column chromatography can also be carried out, which makes it possible to dispense with triethylamine as base. Such a purification is typically not carried out in the case of PEG-modified amidites, however, because they contain the acid-labile DMT protecting group.

For the coupling reaction of the lipid-modified phosphoramidites to the 5′ end of the nucleic acid used according to the invention there are preferably used those solvents in which the amidites used are sufficiently soluble. For example, owing to the high lipophilicity of the amidites used according to the invention, their solubility in acetonitrile can be limited. Apart from acetonitrile as the solvent that is typically used, a solution of chlorinated hydrocarbons is therefore preferably used for the coupling reactions, for example a 0.1 M solution in (absolute) dichloromethane. The use of dichloromethane requires some changes to the standard protocol of the synthesis cycle, however. For example, in order to avoid precipitation of the amidite in the pipes of the automatic synthesis device and on the carrier material, all the valves and pipes that come into contact with the amidite are flushed with (absolute) dichloromethane before and after the actual coupling step and blown dry.

When lipid-modified amidites are used, high coupling yields are typically obtained, which are comparable with the coupling yield of amidites conventionally used in the prior art. The kinetics of the reaction of lipid-modified amidites generally proceeds more slowly. For this reason, the coupling times are preferably (markedly) lengthened when lipid-modified amidites are used, as compared with standard protocols. Such coupling times can easily be determined by a person skilled in the art. Because a capping step after the coupling can be omitted, it is likewise possible, if required, to carry out a further synthesis cycle with the same lipid-modified amidite, in order to increase the overall yield of the reaction. In this case, the detritylation step is usually not carried out, for example in the case of DMT-modified lipids such as DMT-PEG.

In the synthesis of 5′-lipid-modified nucleic acids according to the invention, the phosphite triester via which the lipid is bound to the nucleic acid can be oxidised by a sulfurising agent. To this end there is preferably used a sulfurising agent that achieves oxidation of the phosphotriester as completely as possible. Otherwise, the sulfurisation reaction, for example for steric reasons, may proceed so incompletely that only a small amount of product, or no product at all, is obtained after the ammoniacal cleavage and deprotection of the MON. This phenomenon is dependent on the type of modification, the sulfurising agent used and the sulfurisation conditions. The oxidation is therefore carried out preferably with iodine. As a result, although a phosphodiester bond is introduced, it is not to be expected, owing to the proximity of the lipid radical, that this bond will be recognised as a substrate by nucleases.

The linkers or (bifunctional) lipids contained in the lipid-modified nucleic acid used according to the invention, or optionally the nucleic acids used, can, as described hereinbefore, be coupled directly or indirectly to a carrier material. Direct coupling is carried out preferably directly with the carrier material, while indirect coupling to the carrier material is typically carried out via a further (coupling) molecule. The bond formed by the coupling to a carrier material preferably exhibits a (cleavable) covalent bond with the linker or bifunctional lipid and/or a (cleavable) covalent bond with the solid phase. Compounds suitable as (coupling) molecule are, for example, dicarboxylic acids, for example succinyl radicals (=succinyl anchors), oxalyl radicals (=Oxalyl anchors), etc. Linkers, (bifunctional) lipids or optionally used nucleic acids which, like, for example, aminoalkyl radicals (e.g. aminopropyl or aminohexanyl radicals), carrying a free amino function, can be bound to the carrier material via a phthalimide linker. Thiol-containing linkers, (bifunctional) lipids or optionally used nucleic acids can be bound in disulfide form to the carrier material. Suitable carrier materials in connection with this invention are in particular solid phases such as CPG, Tentagel®, amino-functionalised PS-PEG (Tentagel® S NH₂), etc., preferably Tentagel® or amino-functionalised PS-PEG (Tentagel® S NH₂). According to a particular embodiment it is possible for the coupling to a carrier material to couple, for example, the succinates of the described linkers or bifunctional lipids used according to the invention, with TBTU/NMM (1H-benzotriazol-1-yl-1,1,3,3-tetramethyluronium tetrafluoroborate/N-methylmorpholine) as coupling reagent, to amino-functionalised PS-PEG (Tentagel® S NH₂). In the case of PS-PEG carrier materials on the 1 μmol scale that is conventionally used, the best results are typically obtained with loads of from 50 to 100 μmol/g (E. Bayer, K. Bleicher, M. Maier Z. Naturforsch. 50b (1995) 1096). If, however, nucleotides are to be synthesised on a large scale according to the invention, the loading of the carrier materials is preferably as high as possible (≧100 μmol). According to the invention, such a process likewise results in good coupling yields (M. Gerster, M. Maier, N. Clausen, J. Schewitz, E. Bayer Z. Naturforsch. 52b (1997)110). For example, carrier materials such as, for example, resins with a load of up to 138 μmol/g or optionally more can be used with good synthesis yields. Because the coupling yields with the above-described linkers or bifunctional lipids are approximately 100%, the loading of the carrier material can be adjusted relatively precisely via the stoichiometry of these compounds. The loading is preferably monitored by spectroscopic quantification of the cleaved DMT protecting group (see experimental part). The residual amino functions still present on the carrier material can be capped with acetic anhydride. This capping is normally carried out following the loading of the carrier material but can also take place directly in the nucleic acid synthesis, for example in a DNA synthesiser. For the synthesis of lipid-modified nucleic acids on the derivatised PS-PEG carrier materials there are preferably used synthesis cycles developed specifically for Tentagel®, which take into account the characteristic properties of the material (E. Bayer, M. Maier, K. Bleicher, H.-J. Gaus Z. Naturforsch. 50b (1995) 671, E. Bayer, K. Bleicher, M. Maier Z. Naturforsch. 50b (1995) 1096.). Preferred changes as compared with the standard protocol include: lengthened reaction times in the coupling, capping and oxidation steps; increased number of detritylation steps; lengthened washing steps after each step; and use of an ascorbic-acid-containing washing solution (0.1 M in dioxane/water=9:1) after the oxidation step that is usually necessary (for oxidation of the phosphite triester) during the amidite process, in order to remove traces of iodine.

It should be noted that the nature of the modification can have an influence on the individual steps of the synthesis cycle. For example, in the case of PEG₁₅₀₀-derivatised carrier materials, a considerably slowed reaction kinetics is observed, which requires the detritylation steps to be lengthened again and the coupling time to be lengthened in addition. Such changes and adaptations are within the scope of the normal capability of a person skilled in the art and can be carried out at any time within the context of the present disclosure. With these reaction cycles so modified, both lipid-modified phosphorodiesters and phosphorothioates can be synthesised. The coupling yields of amidites on linkers or bifunctional lipids used according to the invention are not impaired by the lipid radicals but correspond to conventional values (97-99%). The possibility of 5′ derivatisation and the introduction of further modifications, for example at base, sugar or phosphate backbone, is retained when such 3′ modifications are used.

According to a further embodiment, the immune-stimulating adjuvant according to the invention can be combined with further adjuvants known in the prior art.

The present invention relates also to pharmaceutical compositions containing an immune-stimulating adjuvant as described above, at least one active ingredient and optionally a pharmaceutically acceptable carrier and/or further auxiliary substances and additives and/or adjuvants.

The pharmaceutical compositions according to the present invention typically comprise a safe and effective amount of the immune-stimulating adjuvant according to the invention. As used here, “safe and effective amount” means an amount of a compound that is sufficient to significantly induce a positive modification of a condition to be treated, for example of a tumour or infectious disease. At the same time, however, a “safe and effective amount” is small enough to avoid serious side-effects, that is to say to permit a sensible relationship between advantage and risk. The determination of these limits typically lies within the scope of sensible medical judgment. In relation to the immune-stimulating adjuvant according to the invention, the expression “safe and effective amount” preferably means an amount that is suitable for stimulating the immune system in such a manner that no excessive or damaging immune reactions are achieved but, preferably, also no such immune reactions below a measurable level. A “safe and effective amount” of an adjuvant according to the invention or of an adjuvant according to the invention will vary in connection with the particular condition to be treated and also with the age and physical condition of the patient to be treated, the severity of the condition, the duration of the treatment, the nature of the accompanying therapy, of the particular pharmaceutically acceptable carrier used, and similar factors, within the knowledge and experience of the accompanying doctor. The pharmaceutical compositions according to the invention can be used according to the invention for human and also for veterinary medical purposes.

In addition to the immune-stimulating adjuvant according to the invention, the pharmaceutical composition according to the invention preferably contains at least one active ingredient. An active ingredient in this connection is a compound that has a therapeutic effect against a particular indication, preferably cancer diseases or infectious diseases. Such compounds include, without implying any limitation, peptides, proteins, nucleic acids, (therapeutically active) low molecular weight organic or inorganic compounds (molecular weight less than 5000, preferably less than 1000), sugars, antigens or antibodies, therapeutic agents already known in the prior art, etc. According to a particular embodiment, the above-described immune-stimulating adjuvant according to the invention can itself be an active ingredient. This is the case in particular when the lipid of the lipid-modified nucleic acid is a therapeutically active molecule, such as, for example, a vitamin, or steroid, as described above, for example α-tocopherol (vitamin E), D-α-tocopherol, L-α-tocopherol, D,L-α-tocopherol, vitamin E succinate (VES), vitamin A and its derivatives, vitamin D and its derivatives, vitamin K and its derivatives, etc.

According to a first embodiment, the active ingredient contained in the pharmaceutical composition according to the invention is in the form of an antigen or immunogen. An “antigen” or “immunogen” is to be understood as being any structure that is able to bring about the formation of antibodies and/or the activation of a cellular immune response. According to the invention, therefore, the terms “antigen” and “immunogen” are used synonymously. Examples of antigens are peptides, polypeptides, that is to say also proteins, cells, cell extracts, polysaccharides, polysaccharide conjugates, lipids, glycolipids and carbohydrates. There come into consideration as antigens, for example, tumour antigens, viral, bacterial, fungal and protozoological antigens. Preference is given to surface antigens of tumour cells and surface antigens, in particular secreted forms, of viral, bacterial, fungal or protozoological pathogens. The antigen can, of course, be present, for example, in a vaccine according to the invention, also in the form of a haptene coupled to a suitable carrier.

The active ingredient contained in the pharmaceutical composition according to the invention can be present according to a second embodiment in the form of an antibody. In this connection, any therapeutically suitable antibody can be used. Particular preference is given according to the invention to an antibody directed against antigens, proteins or nucleic acids that play an important part in cancer diseases or infectious diseases, for example cell surface proteins, tumour suppressor genes or inhibitors thereof, growth and elongation factors, apoptosis-relevant proteins, tumour antigens, or antigens as described hereinbefore, etc.

According to a third embodiment, the active ingredient contained in the pharmaceutical composition according to the invention is in the form of a nucleic acid. Such a nucleic acid can be single-stranded or double-stranded and can be in the form of a homo- or hetero-duplex and also in linear or circular form. A nucleic acid contained as active ingredient in the pharmaceutical composition is not limited in terms of its length and can include any naturally occurring nucleic acid sequence or its complement or a fragment thereof. Likewise, the nucleic acid used in this connection can be partially or wholly of synthetic nature. For example, the nucleic acid can include a nucleic acid that codes for a (therapeutically relevant) protein and/or that is capable of bringing about an immune reaction, for example an antigen or a nucleic acid coding for an antigen. An antigen here is preferably an antigen as described hereinbefore.

According to a first preferred alternative of the above-mentioned embodiment, the nucleic acid contained as active ingredient in the pharmaceutical composition according to the invention is in the form of mRNA. Such a mRNA can be added in its naked form to the pharmaceutical composition according to the invention or in a stabilised form that reduces or even prevents the degradation of the nucleic acid in vivo, for example by exo- and/or endo-nucleases.

For example, the mRNA contained as active ingredient in the pharmaceutical composition according to the invention can be stabilised by an above-defined 5′ cap and/or a poly-A tail at the 3′ end of at least 50 nucleotides, preferably at least 70 nucleotides, more preferably at least 100 nucleotides, particularly preferably at least 200 nucleotides. As already mentioned, the terminal structure is of critical importance in vivo. The RNA is recognised as mRNA via these structures and the degradation is regulated. In addition, however, there are further processes that stabilise or destabilise RNA. Many of these processes are still unknown, but an interaction between the RNA and proteins often appears to be decisive therefor. For example, an “mRNA surveillance system” has recently been described (Hellerin and Parker, Ann. Rev. Genet. 1999, 33: 229 to 260), in which incomplete or non-sense mRNA is recognised by particular feedback protein interactions in the cytosol and is made amenable to degradation, a majority of these processes being carried out by exonucleases.

The stabilisation of the mRNA contained as active ingredient in the pharmaceutical composition according to the invention can likewise be carried out by associating or complexing the mRNA with, or binding it to, a cationic compound, in particular a polycationic compound, for example a (poly)cationic peptide or protein. In particular the use of protamine, nucleoline, spermin or spermidine as the polycationic, nucleic-acid-binding protein is particularly effective. Furthermore, the use of other cationic peptides or proteins, such as poly-L-lysine or histones, is likewise possible. This procedure for stabilising mRNA is described in EP-A-1083232, the disclosure of which is incorporated by reference into the present invention in its entirety. Further preferred cationic substances which can be used for stabilising the mRNA present as active ingredient include cationic polysaccharides, for example chitosan, polybrene, polyethyleneimine (PEI) or poly-L-lysine (PLL), etc. Apart from the action of the lipid-modified nucleic acid according to the invention as adjuvant in improving cell permeability, which is already advantageous, the association or complexing of the mRNA with cationic compounds preferably increases the transfer of the mRNA present as active ingredient into the cells to be treated or into the organism to be treated.

A further possible method of stabilising mRNA that can be present as active ingredient in the pharmaceutical composition according to the invention is the targeted changing of the sequence of the mRNA by removing or changing so-called destabilising sequence elements (DSEs). Signal proteins are able to bind to these destabilising sequence elements (DSEs), which occur in eukaryotic mRNA in particular, and regulate the enzymatic degradation of the mRNA in vivo. Therefore, in order further to stabilise the mRNA present as active ingredient, one or more changes are preferably made as compared with the corresponding region of the wild-type mRNA, so that no destabilising sequence elements are present. Of course, it is likewise preferred according to the invention to eliminate DSEs optionally present in the untranslated regions (3′- and/or 5′-UTR) from the mRNA. Examples of the above DSEs are AU-rich sequences (“AURES”), which occur in 3′-UTR sections of numerous unstable mRNAs (Caput et al., Proc. Natl. Acad. Sci. USA 1986, 83: 1670 to 1674). The mRNA used as active ingredient is therefore preferably changed as compared with the wild-type mRNA in such a manner that it does not contain any such destabilising sequences. This is also true of those sequence motifs that are recognised by possible endonucleases, for example the sequence GAACAAG, which is contained in the 3′-UTR segment of the gene coding for the transferrin receptor (Binder et al., EMBO J. 1994, 13: 1969 to 1980). Such sequence motifs are preferably also eliminated from the lipid-modified nucleic acid according to the invention.

The mRNA optionally present as active ingredient in the pharmaceutical composition according to the invention can further be changed, for example for an efficient translation that may be desired, in such a manner that effective binding of the ribosomes to the ribosomal binding site (Kozak sequence: GCCGCCACCAUGG, the AUG forms the start codon) takes place. It has been noted in this connection that an increased A/U content around this position permits more efficient ribosome binding to the mRNA.

Furthermore, it is possible to introduce one or more so-called IRESs (internal ribosome entry side) into the mRNA used as active ingredient. An IRES can thus function as the only ribosomal binding site, but it can also serve to provide a mRNA that codes for a plurality of peptides or polypeptides which are to be translated independently of one another by the ribosomes (“multicistronic mRNA”). Examples of IRES sequences which can be used according to the invention are those from picorna viruses (e.g. FMDV), plague viruses (CFFV), polio viruses (PV), encephalo-myocarditis viruses (ECMV), foot-and-mouth viruses (FMDV), hepatitis C viruses (HCV), conventional swine fever viruses (CSFV), murine leukoma virus (MLV), simean immune deficiency virus (SIV) or cricket paralysis viruses (CrPV).

The mRNA optionally used as active ingredient in the pharmaceutical composition according to the invention can likewise contain in its 5′- and/or 3′-untranslated regions stabilising sequences that are capable of increasing the half-life of the mRNA in the cytosol. These stabilising sequences can exhibit 100% sequence homology with naturally occurring sequences that occur in viruses, bacteria and eukaryotes, but they can also be partially or wholly of synthetic nature. As examples of stabilising sequences which can be used in the present invention there may be mentioned the untranslated sequences (UTR) of the β-globin gene, for example of Homo sapiens or Xenopus laevis. Another example of a stabilising sequence has the general formula (C/U)CCANₓCCC(U/A)PyₓUC(C/U)CC, which is contained in the 3′-UTR of the very stable mRNA that codes for α-globin, α-(I)-collagen, 15-lipoxygenase or for tyrosine-hydroxylase (see Holcik et al., Proc. Natl. Acad. Sci. USA 1997, 94: 2410 to 2414). Of course, such stabilising sequences can be used individually or in combination with one another as well as in combination with other stabilising sequences known to a person skilled in the art.

In order further to increase an optionally desired translation, the mRNA used as active ingredient can exhibit the following modifications as compared with a corresponding wild-type mRNA, which modifications can be present either as alternatives or in combination with one another. On the one hand, the G/C content of the region of the modified mRNA coding for a peptide or polypeptide can be greater than the G/C content of the coding region of the wild-type mRNA coding for the peptide or polypeptide, the amino acid sequence coded for being unchanged compared with the wild type. This modification is based on the fact that, for an efficient translation of an mRNA, the sequence of the region of the mRNA to be translated is critical. The composition and sequence of the various nucleotides plays a large part thereby. In particular, sequences having an increased G(guanosine)/C(cytosine) content are more stable than sequences having an increased A(adenosine)/U(uracil) content. According to the invention, therefore, while retaining the translated amino acid sequence, the codons are varied as compared with the wild-type mRNA in such a manner that they contain more G/C nucleotides. Because several codons code for the same amino acid (degeneracy of the genetic code), the codons that are advantageous for the stability can be determined (alternative codon usage). In dependence on the amino acid to be coded for by the mRNA, different possibilities for the modification of the mRNA sequence as compared to the wild-type sequence are possible. In the case of amino acids coded for by codons that contain solely G or C nucleotides, no modification of the codon is necessary. Accordingly, the codons for Pro (CCC or CCG), Arg (CGC or CGG), Ala (GCC or GCG) and Gly (GGC or GGG) do not require any change because no A or U is present. In the following cases, the codons that contain A and/or U nucleotides are changed by the substitution of different codons that code for the same amino acids but do not contain A and/or U. Examples are: the codons for Pro can be changed from CCU or CCA to CCC or CCG; the codons for Arg can be changed from CGU or CGA or AGA or AGG to CGC or CGG; the codons for Ala can be changed from GCU or GCA to GCC or GCG; the codons for Gly can be changed from GGU or GGA to GGC or GGG. In other cases, although A and U nucleotides cannot be eliminated from the codons, it is possible to reduce the A and U content by the use of codons that contain fewer A and/or U nucleotides. For example: the codons for Phe can be changed from UUU to UUC; the codons for Leu can be changed from UUA, CUU or CUA to CUC or CUG; the codons for Ser can be changed from UCU or UCA or AGU to UCC, UCG or AGC; the codon for Tyr can be changed from UAU to UAC; the stop codon UAA can be changed to UAG or UGA; the codon for Cys can be changed from UGU to UGC; the codon His can be changed from CAU to CAC; the codon for Gln can be changed from CAA to CAG; the codons for Ile can be changed from AUU or AUA to AUC; the codons for Thr can be changed from ACU or ACA to ACC or ACG; the codon for Asn can be changed from AAU to AAC; the codon for Lys can be changed from AAA to AAG; the codons for Val can be changed from GUU or GUA to GUC or GUG; the codon for Asp can be changed from GAU to GAC; the codon for Glu can be changed from GAA to GAG. In the case of the codons for Met (AUG) and Trp (UGG), on the other hand, there is no possibility of sequence modification. The substitutions listed above can, of course, be used individually but also in all possible combinations for increasing the G/C content of the modified mRNA as compared with the original sequence. Thus, for example, all codons for Thr occurring in the original (wild-type) sequence can be changed to ACC (or ACG). Preferably, however, combinations of the above substitution possibilities are used, for example: substitution of all codons in the original sequence coding for Thr to ACC (or ACG) and substitution of all codons originally coding for Ser to UCC (or UCG or AGC); substitution of all codons in the original sequence coding for Ile to AUC and substitution of all codons originally coding for Lys to AAG and substitution of all codons originally coding for Tyr to UAC; substitution of all codons in the original sequence coding for Val to GUC (or GUG) and substitution of all codons originally coding for Glu to GAG and substitution of all codons originally coding for Ala to GCC (or GCG) and substitution of all codons originally coding for Arg to CGC (or CGG); substitution of all codons in the original sequence coding for Val to GUC (or GUG) and substitution of all codons originally coding for Glu to GAG and substitution of all codons originally coding for Ala to GCC (or GCG) and substitution of all codons originally coding for Gly to GGC (or GGG) and substitution of all codons originally coding for Asn to AAC; substitution of all codons in the original sequence coding for Val to GUC (or GUG) and substitution of all codons originally coding for Phe to UUC and substitution of all codons originally coding for Cys to UGC and substitution of all codons originally coding for Leu to CUG (or CUC) and substitution of all codons originally coding for Gln to CAG and substitution of all codons originally coding for Pro to CCC (or CCG); etc. Preferably, the G/C content of the region (or of each other further section optionally present) of the mRNA that codes for the peptide or polypeptide is increased by at least 7% points, more preferably by at least 15% points, particularly preferably by at least 20% points, as compared with the G/C content of the coded region of the wild-type mRNA coding for the corresponding peptide or polypeptide. It is particularly preferred in this connection to increase the G/C content of the mRNA so modified in comparison with the wild-type sequence to the maximum possible degree.

A further preferred modification of a mRNA used as active ingredient in the pharmaceutical composition is based on the finding that the translation efficiency is also determined by a different frequency in the occurrence of tRNAs in cells. If, therefore, so-called “rare” codons are present in an increased number in a RNA sequence, then the corresponding mRNA is translated markedly more poorly than in the case where codons coding for relatively “frequent” tRNAs are present. According to the invention, therefore, the coding region in the mRNA used as active ingredient is changed as compared with the corresponding region of the wild-type mRNA in such a manner that at least one codon of the wild-type sequence that codes for a relatively rare tRNA in the cell is replaced by a codon that codes for a relatively frequent tRNA in the cell, which carries the same amino acid as the relatively rare tRNA. By means of this modification, the RNA sequences are so modified that codons are introduced for which frequently occurring tRNAs are available. Which tRNAs occur relatively frequently in the cell and which, by contrast, are relatively rare is known to a person skilled in the art; see, for example, Akashi, Curr. Opin. Genet. Dev. 2001, 11(6): 660-666. By means of this modification it is possible according to the invention to replace all codons of the wild-type sequence that code for a relatively rare tRNA in the cell by in each case a codon that codes for a relatively frequent tRNA in the cell, which in each case carries the same amino acid as the relatively rare tRNA. It is particularly preferred to link the increased, in particular maximum, sequential G/C portion in the mRNA as described above with the “frequent” codons, without changing the amino acid sequence of an antigenic peptide or polypeptide (one or more) coded for by the coding region of the mRNA.

According to a second preferred alternative of the last-mentioned embodiment, the nucleic acid contained as active ingredient in the pharmaceutical composition according to the invention is in the form of dsRNA, preferably siRNA. A dsRNA, or an siRNA, is of interest particularly in connection with the phenomenon of RNA interference. Attention was drawn to the phenomenon of RNA interference in the course of immunological research. In recent years, a RNA-based defence mechanism has been discovered, which occurs both in the kingdom of the fungi and in the plant and animal kingdom and acts as an “immune system of the genome”. The system was originally described in various species independently of one another, first in C. elegans, before it was possible to identify the underlying mechanisms of the processes as being identical: RNA-mediated virus resistance in plants, PTGS (posttranscriptional gene silencing) in plants, and RNA interference in eukaryotes are accordingly based on a common procedure. The in vitro technique of RNA interference (RNAi) is based on double-stranded RNA molecules (dsRNA), which trigger the sequence-specific suppression of gene expression (Zamore (2001) Nat. Struct. Biol. 9: 746-750; Sharp (2001) Genes Dev. 5:485-490: Hannon (2002) Nature 41: 244-251). In the transfection of mammalian cells with long dsRNA, the activation of protein kinase R and RnaseL brings about unspecific effects, such as, for example, an interferon response (Stark et al. (1998) Annu. Rev. Biochem. 67: 227-264; He and Katze (2002) Viral Immunol. 15: 95-119). These unspecific effects are avoided when shorter, for example 21- to 23-mer, so-called siRNA (small interfering RNA), is used, because unspecific effects are not triggered by siRNA that is shorter than 30 bp (Elbashir et al. (2001) Nature 411: 494-498). Recently, dsRNA molecules have also been used in vivo (McCaffrey et al. (2002), Nature 418: 38-39; Xia et al. (2002), Nature Biotech. 20: 1006-1010; Brummelkamp et al. (2002), Cancer Cell 2: 243-247).

The double-stranded RNA (dsRNA) used as active ingredient therefore preferably contains a sequence having the general structure 5′-(N₁₇₋₂₉)-3′, wherein N is any base and represents nucleotides. The general structure is composed of a double-stranded RNA having a macromolecule composed of ribonucleotides, the ribonucleotide consisting of a pentose (ribose), an organic base and a phosphate. The organic bases in the RNA here consist of the purine bases adenine (A) and guanine (G) and of the pyrimidine bases cytosine (C) and uracil (U). The dsRNA used as active ingredient according to the invention contains such nucleotides or nucleotide analogues having an oriented structure. The dsRNAs used as active ingredient according to the invention preferably have the general structure 5′-(N₁₉₋₂₅)-3′, more preferably 5′-(N₁₉₋₂₄)-3′, yet more preferably 5′-(N₂₁₋₂₃)-3′, wherein N is any base. Preferably at least 90%, more preferably 95% and especially 100% of the nucleotides of a dsRNA used as active ingredient will be complementary to a section of the mRNA sequence of a (therapeutically relevant) protein or antigen described (as active ingredient) hereinbefore. 90% complementary means that with a length of a dsRNA used according to the invention of, for example, 20 nucleotides, this contains not more than 2 nucleotides without corresponding complementarity with the corresponding section of the mRNA. The sequence of the double-stranded RNA used according to the invention is, however, preferably wholly complementary in its general structure with a section of the mRNA of a protein or antigen described as active ingredient hereinbefore.

In principle, all the sections having a length of from 17 to 29, preferably from 19 to 25, base pairs that occur in the coding region of the mRNA can serve as target sequence for a dsRNA used as active ingredient according to the invention. Equally, dsRNAs used as active ingredient can also be directed against nucleotide sequences of a (therapeutically relevant) protein or antigen described (as active ingredient) hereinbefore that do not lie in the coding region, in particular in the 5′ non-coding region of the mRNA, for example, therefore, against non-coding regions of the mRNA having a regulatory function. The target sequence of the dsRNA used as active ingredient of a protein or antigen described hereinbefore can therefore lie in the translated and untranslated region of the mRNA and/or in the region of the control elements. The target sequence of a dsRNA used as active ingredient can also lie in the overlapping region of untranslated and translated sequence; in particular, the target sequence can comprise at least one nucleotide upstream of the start triplet of the coding region of the mRNA.

A modified nucleotide can preferably occur in a dsRNA present as active ingredient in the pharmaceutical composition according to the invention. The expression “modified nucleotide” means according to the invention that the nucleotide in question has been chemically modified. The person skilled in the art understands by the expression “chemical modification” that the modified nucleotide has been changed in comparison with naturally occurring nucleotides by the replacement, addition or removal of one or more atoms or atom groups. At least one modified nucleotide in the dsRNA used according to the invention serves on the one hand for stability and on the other hand to prevent dissociation. Preferably from 2 to 10 and more preferably from 2 to 5 nucleotides in a dsRNA used according to the invention have been modified. Advantageously, at least one 2′-hydroxy group of the nucleotides of the dsRNA in the double-stranded structure has been replaced by a chemical group, preferably at 2′-amino or a 2′-methyl group. At least one nucleotide in at least one strand of the double-stranded structure can also be a so-called “locked nucleotide” having a sugar ring that has been chemically modified, preferably by a 2′-O, 4′-C-methylene bridge. Several nucleotides of the dsRNA used according to the invention are advantageously locked nucleotides. Moreover, by modification of the backbone of a dsRNA used according to the invention, premature degradation thereof can be prevented. Particular preference is given in this connection to a dsRNA that has been modified in the form of phosphorothioate, 2′-O-methyl-RNA, LNA, LNA/DNA gapmers, etc. and therefore has a longer half-life in vivo.

The ends of the double-stranded RNA (dsRNA) used as active ingredient in the pharmaceutical composition according to the invention can preferably be modified in order to counteract degradation in the cell or dissociation in the individual strands, in particular in order to avoid premature degradation by nucleases. A normally undesirable dissociation of the individual strands of dsRNA occurs in particular when low concentrations thereof or short chain lengths are used. For the particularly effective inhibition of dissociation, the cohesion, effected by the nucleotide pairs, of the double-stranded structure of dsRNA used according to the invention can be increased by at least one, preferably more than one, chemical linkage(s). A dsRNA used as active ingredient according to the invention whose dissociation has been reduced has higher stability towards enzymatic and chemical degradation in the cell or in the organism (in vivo) or ex vivo and therefore has a longer half-life. A further possibility for preventing premature dissociation in the cell of dsRNA used according to the invention consists in that a hairpin loop(s) can be formed at each end of the strands. In a particular embodiment, a dsRNA used according to the invention therefore has a hairpin structure in order to slow the dissociation kinetics. In such a structure, a loop structure is formed preferably at the 5′ and/or 3′ end. Such a loop structure has no hydrogen bridges, and typically therefore no complementarity, between nucleotide bases. Typically, such a loop has a length of at least 5, preferably at least 7 nucleotides and in that manner binds the two complementary individual strands of a dsRNA used according to the invention. In order to prevent dissociation of the strands, the nucleotides of the two strands of the dsRNA used according to the invention can likewise preferably be so modified that strengthening of the hydrogen bridge bond is achieved, for example by increasing the hydrogen bridge bond capacity between the bases by optionally modified nucleotides. As a result, the stability of the interaction between the strands is increased and the dsRNA is protected against attack by RNases.

According to a particularly preferred embodiment, the dsRNA used as active ingredient in the pharmaceutical composition according to the invention is directed against the mRNA of a protein or antigen as described hereinbefore. The dsRNA used preferably thereby suppresses the translation of an above-described protein or antigen in a cell to the extent of at least 50%, more preferably 60%, yet more preferably 70% and most preferably at least 90%, that is to say the cell contains preferably not more than half of the naturally occurring (without treatment with dsRNA used according to the invention) cellular concentration of an above-described protein or antigen. The suppression of the translation of these proteins or antigens in cells after addition of dsRNA molecules used according to the invention is based on the phenomenon of RNA interference caused by such molecules. The dsRNA used according to the invention is then so-called siRNA, which triggers the phenomenon of RNA interference and can bind the mRNA of an above-described protein or antigen. Measurement or demonstration of the translation suppression triggered in cells by the dsRNA used according to the invention can be carried out by Northern blot, quantitative real-time PCR or, at protein level, with specific antibodies against an above-described protein or antigen. The dsRNA optionally used as active ingredient in the pharmaceutical composition according to the invention, and a corresponding siRNA, can be prepared by processes known to the person skilled in the art.

In order further to increase the immunogenity, the pharmaceutical composition according to the invention can additionally contain one or more auxiliary substances. A synergistic action of the immune-stimulating adjuvant according to the invention and of an auxiliary substance optionally additionally contained in the pharmaceutical composition and/or optionally of an active ingredient as described above is preferably achieved thereby. Depending on the various types of auxiliary substances, various mechanisms can come into consideration in this respect. For example, compounds that permit the maturation of dendritic cells (DCs), for example lipopolysaccharides, TNF-α or CD40 ligand, form a first class of suitable auxiliary substances. In general, it is possible to use as auxiliary substance any agent that influences the immune system in the manner of a “danger signal” (LPS, GP96, etc.) or cytokines, such as GM-CFS, which allow an immune response produced by the immune-stimulating adjuvant according to the invention to be enhanced and/or influenced in a targeted manner. Particularly preferred auxiliary substances are cytokines, such as monokines, lymphokines, interleukins or chemokines, for example IL-1, IL-2, IL-3, IL-4, IL-5, IL-6, IL-7, IL-8, IL-9, IL-10, IL-12, INF-γ, INF-β, GM-CFS, M-CSF, G-CSF, LT-β, TNF-α, or interferons, for example IFN-γ, or growth factors, for example hGH.

The pharmaceutical composition according to the invention can also additionally contain an adjuvant known in the prior art. In connection with the present invention, adjuvants known in the prior art include, without implying any limitation, aluminium hydroxide, (complete or incomplete) Freund's adjuvant, and also above-described stabilising cationic peptides or polypeptides, such as protamine, nucleoline, spermine or spermidine, and cationic polysaccharides, in particular chitosan, TDM, MDP, muramyl dipeptide, alum solution, pluronics, etc. Furthermore, lipopeptides, such as Pam3Cys, are also particularly suitable for combining with the immune-stimulating adjuvant according to the invention (see Deres et al., Nature 1989, 342: 561-564).

The pharmaceutical composition according to the invention can optionally contain a pharmaceutically acceptable carrier. The expression “pharmaceutically acceptable carrier” used here preferably includes one or more compatible solid or liquid fillers or diluents or encapsulating compounds, which are suitable for administration to a person. The term “compatible” as used here means that the constituents of the composition are capable of being mixed with the active ingredient, with the adjuvant as such and with one another in such a manner that no interaction occurs which would substantially reduce the pharmaceutical effectiveness of the composition under usual use conditions. Pharmaceutically acceptable carriers must, of course, have sufficiently high purity and sufficiently low toxicity to make them suitable for administration to a person to be treated. Some examples of compounds which can be used as pharmaceutically acceptable carriers or constituents thereof are sugars, for example lactose, glucose and sucrose; starches, such as, for example, corn starch or potato starch; cellulose and its derivatives, such as, for example, sodium carboxymethylcellulose, ethylcellulose, cellulose acetate; powdered tragacanth; malt; gelatin; tallow; solid glidants, such as, for example, stearic acid, magnesium stearate; calcium sulfate; vegetable oils, such as, for example, groundnut oil, cottonseed oil, sesame oil, olive oil, corn oil and oil from theobroma; polyols, such as, for example, polypropylene glycol, glycerol, sorbitol, mannitol and polyethylene glycol; alginic acid; emulsifiers, such as, for example, Tween®; wetting agents, such as, for example, sodium lauryl sulfate; colouring agents; taste-imparting agents, pharmaceutical carriers; tablet-forming agents; stabilisers; antioxidants; preservatives; pyrogen-free water; isotonic saline and phosphate-buffered solutions.

The choice of a pharmaceutically acceptable carrier is determined in principle by the manner in which the pharmaceutical compositions according to the invention are administered. The pharmaceutical compositions according to the invention can be administered, for example, systemically. Routes for administration include, for example, transdermal, oral, parenteral, including subcutaneous or intravenous injections, topical and/or intranasal routes. The suitable amount of the pharmaceutical composition to be used can be determined by routine experiments with animal models. Such models include, without implying any limitation, rabbit, sheep, mouse, rat, dog and non-human primate models. Preferred unit dose forms for injection include sterile solutions of water, physiological saline or mixtures thereof. The pH of such solutions should be adjusted to about 7.4. Suitable carriers for injection include hydrogels, devices for controlled or delayed release, polylactic acid and collagen matrices. Suitable pharmaceutically acceptable carriers for topical application include those which are suitable for use in lotions, creams, gels and the like. If the compound is to be administered perorally, tablets, capsules and the like are the preferred unit dose form. The pharmaceutically acceptable carriers for the preparation of unit dose forms which can be used for oral administration are well known in the prior art. The choice thereof will depend on secondary considerations such as taste, costs and storability, which are not critical for the purposes of the present invention, and can be made without difficulty by a person skilled in the art.

According to a particular embodiment, the pharmaceutical composition can also be present in the form of a vaccine. Vaccines according to the invention typically comprise a composition as described above for the pharmaceutical composition, the composition of such vaccines according to the invention being determined in particular by the manner in which they are administered. Vaccines according to the invention are preferably administered systemically. Routes for the administration of such vaccines typically include transdermal, oral, parenteral, including subcutaneous or intravenous injections, topical and/or intranasal routes. Vaccines are therefore formulated preferably in liquid or solid form. It is also possible for further auxiliary substances that further increase the immunogenity of the vaccine to be incorporated into a vaccine according to the invention. Advantageously, one or more further such auxiliary substances as defined hereinbefore is/are to be chosen, depending on the immunogenity and other properties of the active ingredient in the vaccine according to the invention.

According to a further preferred object of the present invention, the pharmaceutical compositions according to the invention, particularly preferably the vaccines according to the invention, are used for the treatment of indications mentioned by way of example hereinbelow. With pharmaceutical compositions according to the invention, particularly preferably vaccines according to the invention, it is possible to treat, for example, diseases or conditions that are associated with various pathologically absent immune responses or that require an immune response, preferably an increased immune response, within the context of a therapy. Pharmaceutical compositions or vaccines according to the invention are preferably used to trigger tumour-specific or pathogen-specific immune responses. Such pharmaceutical compositions or vaccines according to the invention can be used particularly preferably for increasing immune responses of antigen-presenting cells (APCs). Likewise particularly preferably, the pharmaceutical compositions or vaccines according to the invention can be used for the treatment of cancer or tumour diseases, preferably selected from colon carcinomas, melanomas, renal carcinomas, lymphomas, acute myeloid leukaemia (AML), acute lymphoid leukaemia (ALL), chronic myeloid leukaemia (CML), chronic lymphocytic leukaemia (CLL), gastrointestinal tumours, pulmonary carcinomas, gliomas, thyroid tumours, mammary carcinomas, prostate tumours, hepatomas, various virus-induced tumours such as, for example, papilloma virus-induced carcinomas (e.g. cervical carcinoma), adenocarcinomas, herpes virus-induced tumours (e.g. Burkitt's lymphoma, EBV-induced B-cell lymphoma), heptatitis B-induced tumours (hepatocell carcinoma), HTLV-1- and HTLV-2-induced lymphomas, acoustic neuromas, cervical cancer, lung cancer, pharyngeal cancer, anal carcinomas, glioblastomas, lymphomas, rectal carcinomas, astrocytomas, brain tumours, stomach cancer, retinoblastomas, basaliomas, brain metastases, medulloblastomas, vaginal cancer, pancreatic cancer, testicular cancer, melanomas, thyroidal carcinomas, bladder cancer, Hodgkin's syndrome, meningiomas, Schneeberger disease, bronchial carcinomas, hypophysis tumour, Mycosis fungoides, oesophageal cancer, breast cancer, carcinoids, neurinomas, spinaliomas, Burkitt's lymphomas, laryngeal cancer, renal cancer, thymomas, corpus carcinomas, bone cancer, non-Hodgkin's lymphomas, urethral cancer, CUP syndrome, head/neck tumours, oligodendrogliomas, vulval cancer, intestinal cancer, colon carcinomas, oesophageal carcinomas, warts, tumours of the small intestine, craniopharyngeomas, ovarian carcinomas, genital tumours, ovarian cancer, liver cancer, pancreatic carcinomas, cervical carcinomas, endometrial carcinomas, liver metastases, penile cancer, tongue cancer, gall bladder cancer, leukaemia, plasmocytomas, uterine cancer, lid tumour, prostate cancer, etc. It is particularly preferred in this connection if the lipid used in the lipid-modified nucleic acid or as active ingredient is α-tocopherol (vitamin E), D-α-tocopherol, L-α-tocopherol, D,L-α-tocopherol or vitamin E succinate (VES). α-Tocopherol (vitamin E) is not very toxic and exhibits potent anti-tumour activity (A. Bendich, L. J. Machlin Am. J. Clin. Nutr. 48 (1988) 612), which makes it appear very promising in cancer therapy. As an explanation for the inhibition of the proliferation of tumour cells or the cytotoxic activity thereon, two mechanisms inter alia are known: On the one hand, vitamin E is a potent antioxidant and a good radical acceptor (C. Borek Ann. NY Acad. Sci. 570 (1990) 417); on the other hand, it is able, by stimulating the immune response, to prevent tumour growth (G. Shklar, J. Schwartz, D. P. Trickler, S. Reid J. Oral Pathol. Med. 19 (1990) 60). In more recent works, a connection has further been found between the expression of the tumour suppressor gene p53 in tumour cells (oral squamous cancer) and treatment with vitamin E succinate (VES) (J. Schwartz, G. Shklar, D. Trickler Oral Oncol. Europ. J. Cancer 29B (1993) 313). It has thereby been possible to observe both a stimulation of the production of wild-type p53, which acts as a tumour suppressor, and a reduction in mutated p53, which develops oncogenic activity. Interestingly, the biological activity of VES on these tumour cells is dose-dependent in two respects: in physiological doses (0.001 to 50 μmol/l), increasing cell growth is to be observed; in pharmacological doses (100 to 154 μmol/l), cell growth is inhibited. This has been shown in cell culture (T. M. A. Elattar, A. S. Virji Anticancer Res. 19 (1999) 365). It has also been possible to induce apoptosis in various breast cancer cell lines by treatment with VES (W. Yu, K. Israel, Q. Y. Liao, C. M. Aldaz, B. G. Sanders, K. Kline Cancer Res. 59 (1999) 953). The induced apoptosis is initiated via an interaction of Fas ligand and Fas receptor. This is to be particularly emphasised because it has hitherto not been possible to observe such a mechanism in the corresponding cell lines. There are various isomers of vitamin E, which differ in the number and position of the methyl groups on the aromatic ring. In the described works, the biologically most active form of naturally occurring vitamin E, α-tocopherol, was used. This in turn occurs in various stereoisomers, because the molecule contains three optically active centres. The natural form of vitamin E is RRR-α-tocopherol (formerly D-α-tocopherol), but the racemate (D,L-α-tocopherol) is predominantly used nowadays. All the above-mentioned forms of vitamin E are likewise included as lipid within the scope of the present invention.

Likewise particularly preferably, the pharmaceutical compositions according to the invention are used for the treatment of infectious diseases. Without implying any limitation, such infectious diseases are preferably selected from influenza, malaria, SARS, yellow fever, AIDS, Lyme borreliosis, Leishmaniasis, anthrax, meningitis, viral infectious diseases such as AIDS, Condyloma acuminata, hollow warts, Dengue fever, three-day fever, Ebola virus, cold, early summer meningoencephalitis (FSME), flu, shingles, hepatitis, herpes simplex type I, herpes simplex type II, Herpes zoster, influenza, Japanese encephalitis, Lassa fever, Marburg virus, measles, foot-and-mouth disease, mononucleosis, mumps, Norwalk virus infection, Pfeiffer's glandular fever, smallpox, polio (childhood lameness), pseudo-croup, German measles, rabies, warts, West Nile fever, chickenpox, cytomegalic virus (CMV), from bacterial infectious diseases such as miscarriage (prostate inflammation), anthrax, appendicitis, borreliosis, botulism, Camphylobacter, Chlamydia trachomatis (inflammation of the urethra, conjunctivitis), cholera, diphtheria, donavanosis, epiglottitis, typhus fever, gas gangrene, gonorrhoea, rabbit fever, Heliobacter pylori, whooping cough, climatic bubo, osteomyelitis, Legionnaire's disease, leprosy, listeriosis, pneumonia, meningitis, bacterial meningitis, anthrax, otitis media, Mycoplasma hominis, neonatal sepsis (Chorioamnionitis), noma, paratyphus, plague, Reiter's syndrome, Rocky Mountain spotted fever, Salmonella paratyphus, Salmonella typhus, scarlet fever, syphilis, tetanus, tripper, tsutsugamushi disease, tuberculosis, typhus, vaginitis (colpitis), soft chancre, and from infectious diseases caused by parasites, protozoa or fungi, such as amoebiasis, bilharziosis, Chagas disease, athlete's foot, yeast fungus spots, scabies, malaria, onchocercosis (river blindness), or fungal diseases, toxoplasmosis, trichomoniasis, trypanosomiasis (sleeping sickness), visceral Leishmaniosis, nappy dermatitis, schistosomiasis, fish poisoning (Ciguatera), candidosis, cutaneous Leishmaniosis, lambliasis (giardiasis), or sleeping sickness, or from infectious diseases caused by Echinococcus, fish tapeworm, fox tapeworm, canine tapeworm, lice, bovine tapeworm, porcine tapeworm, miniature tapeworm.

The invention relates also to the use of an immune-stimulating adjuvant according to the invention in the preparation of a pharmaceutical composition according to the invention or of a vaccine according to the invention for the treatment of indications described hereinbefore, for example for the treatment of the mentioned tumour and infectious diseases. Alternatively, the invention includes the (therapeutic) use of an immune-stimulating adjuvant according to the invention for the treatment of tumour or infectious diseases, as described hereinbefore.

Likewise included in the present invention are kits containing an immune-stimulating adjuvant according to the invention and/or a pharmaceutical composition according to the invention and/or a vaccine according to the invention as well as, optionally, technical instructions with information on the administration and dosage of the immune-stimulating adjuvant according to the invention and/or of the pharmaceutical composition according to the invention and/or of the vaccine according to the invention.

The present invention is illustrated further hereinbelow by means of figures and examples, which are not intended to limit the subject-matter of the invention thereto.

### EXAMPLES

### Example 1

### Synthesis of 1-(4,4′-dimethoxytrityl)-polyethylene glycol (DMT-PEG₁₅₀₀)

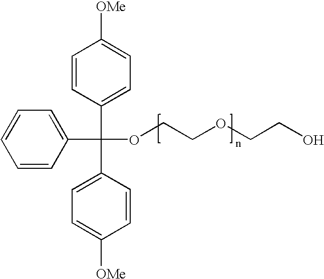

Procedure: 21 g of PEG₁₅₀₀ (14 mmol) are dissolved twice, for drying, in 30 ml of absolute pyridine each time, which is subsequently distilled off azeotropically. The dried starting material is dissolved in 35 ml of absolute pyridine. 4.7 g of 4,4′-dimethoxytrityl chloride (13.9 mmol) dissolved in 35 ml of absolute pyridine are added dropwise to this solution over a period of 30 minutes. Stirring is carried out for a further 2 hours at RT, during which the progress of the reaction is monitored by means of TLC. In addition to detection of the DMT group by means of a UV lamp, the TLC plates can be developed in two steps: 1. in an HCl-saturated atmosphere for the detection of DMT; 2. in an iodine chamber for the detection of PEG; PEG can additionally be detected with Dragendorff-Bürger spray reagent. When the reaction is complete, the solvent is removed and the product is taken up in 50 ml of DCM. The organic phase is washed twice with in each case 25 ml of 5% NaHCO₃ solution and twice with in each case 25 ml of H₂O. Phase separation between aqueous and organic phase is tedious because PEG is of both hydrophobic and hydrophilic nature. After drying over Na₂SO₄, the solvent is removed and the crude product is purified by column chromatography on silica gel with DCM/MeOH/TEA=18:2:0.5. The product-containing fractions are identified by means of TLC, combined and concentrated to dryness. A yellowish oil is obtained which, after thorough drying under a high vacuum, becomes a wax-like solid.

Yield: 18.3 g (72.5% of theory)

TLC (DCM/MeOH/TEA=18:2:0.5): R_{f} value 0.55 (signal spread by the molar mass distribution of PEG)

### Example 2

### Synthesis of 1-(4,4′-dimethoxytrityl)-hexaethylene glycol (DMT-HEG)

Procedure: 10 g of hexaethylene glycol (35 mmol) are dried by coevaporation with 2×30 ml of absolute pyridine and then dissolved in 20 ml of absolute pyridine. Analogously to the procedure of Example 1, the HEG is reacted with 10 g of DMT-Cl (29.5 mmol) dissolved in 50 ml of absolute pyridine. Purification by column chromatography is carried out with ethyl acetate/TEA=95:5. A viscous, yellow oil is obtained as the dried product.

Yield: 12.5 g (60.5% of theory)

TLC (DCM/MeOH=95:5): R_{f} value t=0.59

MS (FD): m/z 583.9 (M⁺)

¹H-NMR (CDCl₃): δ 3.21 (t, DMT-O—CH₂—), 3.47-3.68 (m, —CH₂—), 3.76 (s, —CH₃), 6.77-7.46 (m, aromatic compound)

### Example 3

### Synthesis of 1-(4,4′-dimethoxytrityl)-polyethylene glycol succinate (DMT-PEG-Suc)

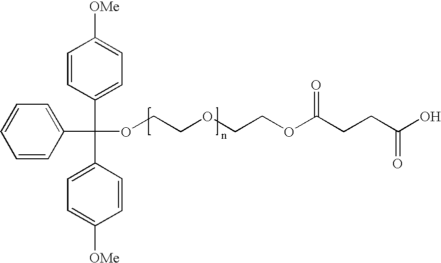

The procedure below can be used for both DMT-PEG₁₅₀₀ and DMT-HEG.

Procedure: 5 g of DMT-PEG₁₅₀₀ (2.8 mmol) are dissolved in 25 ml of DCM/pyridine=5:1, and 420 mg of succinic anhydride (4.2 mmol, i.e. 1.5 eq.) dissolved in 7 ml of pyridine, and 170 mg of DMAP (1.4 mmol, i.e. 0.5 eq.) dissolved in 3 ml of pyridine are added thereto. After 12 hours' stirring at RT, the solvents are removed in vacuo and the residue is taken up in DCM. The organic phase is washed thoroughly three times with NaHCO₃ solution (10% in H₂O) and twice with saturated aqueous NaCl solution, in order to separate off the excess succinic acid. After drying over Na₂SO₄, the solvent is removed. After thorough drying under a high vacuum, the succinates can be used without further working up for coupling to amino-modified carrier materials.

TLC: DMT-PEG₁₅₀₀-Suc (DCM/MeOH/TEA=18:2:0.5): R_{f} value=0.41

DMT-HEG-Suc (DCM/MeOH=9:2): R_{f} value=0.70

### Example 4

### Synthesis of 1-tosyl-2,3-isopropylideneglycerol

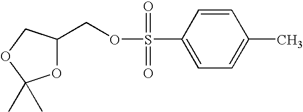

Procedure: 200 mmol of isopropylideneglycerol (26.4 g) are dissolved in 200 ml of acetonitrile, and 22.2 g of triethylamine (220 mmol) are added thereto. 220 mmol of p-toluenesulfonic acid chloride (41.9 g) are dissolved in 250 ml of acetonitrile and added dropwise to the reaction mixture, with stirring, over a period of 2 hours. Stirring is continued for a further 20 hours at RT, whereupon a white precipitate forms, which is filtered off when the reaction is complete. The solvent is removed and the crude product is purified by column chromatography on silica gel with n-hexane/ethyl acetate=2:1. The product-containing fractions are identified by means of TLC, combined and concentrated to dryness. The product is dried under a high vacuum. A yellowish oil is obtained (L. N. Markovskii et al. J. Org. Chem. UdSSR 26 (1990) 2094.).

Yield: 31.3 g (54.7% of theory)

TLC (n-hexane/ethyl acetate=2:1): R_{f} value=0.2

MS (FD): m/z 272.0 (M⁺-CH) (286.3 calculated)

¹H-NMR (CDCl₃): δ 1.31 (s); 1.34 (s); 2.45 (s); 3.74-3.79 (m); 3.90-4.08 (m); 4.23-4.32 (m); 7.36 (d); 7.77 (d)

¹³C-NMR: δ 21.7; 25.2; 26.7 (methyl-C); 66.2; 69.5; 72.9 (glycerol-C); 110.1 (methylene-C); 128.0; 129.9; 132.7; 145.1 (aromatic compound-C)

### Example 5

### Synthesis of 2,3-isopropylidene-1-D,L-α-tocopherolglycerol (Toc1)

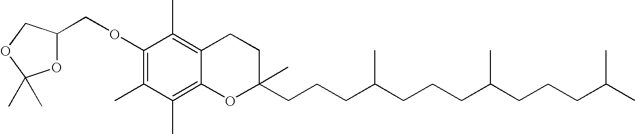

Procedure: 5.6 g of powdered potassium hydroxide (100 mmol) are added to 56 mmol of D,L-α-tocopherol (24.06 g) in 280 ml of DMSO. After 2 hours' stirring at RT with the exclusion of light, 56 mmol of 1-tosyl-2,3-isopropylideneglycerol (16 g) dissolved in 20 ml of DMSO are added dropwise, and stirring is continued for a further 12 hours at 60° C. The reaction mixture is then hydrolysed on 1 litre of ice-water, and the aqueous phase is extracted with 1.5 litres of toluene. After drying over sodium sulfate, the solvent is removed. The crude product is purified by column chromatography on silica gel with n-hexane/ethyl acetate=1:1. The product-containing fractions are identified by means of TLC, combined and concentrated to dryness. The product is dried under a high vacuum and stored with the exclusion of light. A yellowish oil is obtained (D. W. Will, T. Brown Tetrahedron Lett. 33 (1992) 2729.).

Yield: 24.2 g (79.2% of theory)

TLC (n-hexane/ethyl acetate=1:1): R_{f} value=0.69

MS (FD): m/z 544.6 (M⁺)

### Example 6

### Synthesis of 1-D,L-α-tocopherylglycerol (Toc2)

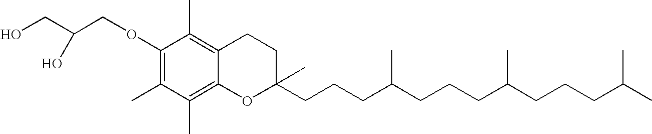

Procedure: 16.9 mmol of Toc1 (9.2 g) are dissolved in 100 ml of HCl (2 M)/THF (1:1) and stirred for 2 hours at RT with the exclusion of light. The solvent is then removed, 2×50 ml of absolute ethanol are added to the residue, and the mixture is concentrated to dryness again. The crude product is purified by column chromatography on silica gel with diethyl ether/toluene=1:1. The product-containing fractions are identified by TLC, combined and concentrated to dryness. The product is dried under a high vacuum and stored with the exclusion of light. A yellow oil is obtained.

Yield: 6.6 g (77.5% of theory)

TLC (diethyl ether/toluene=1:1): R_{f} value=0.22

MS (FD): m/z 504.4 (M⁺)

### Example 7

### Synthesis of [1-(4,4′-dimethoxytrityl)]-3-D,L-α-tocopherylglycerol (Toc3)

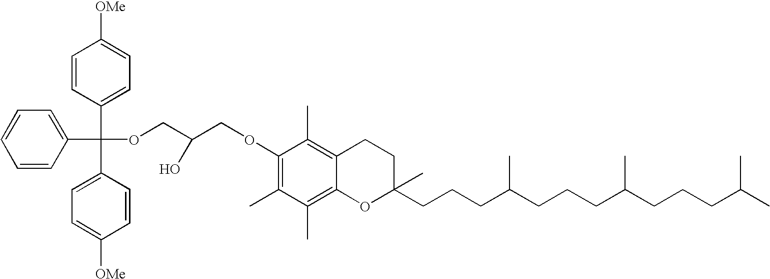

Procedure: 6.6 g of Toc2 (13 mmol) are dissolved twice for drying in 15 ml of absolute pyridine, which is distilled off again azeotropically. The dried starting material is dissolved in 50 ml of absolute pyridine, and 5.34 g of DMT-Cl (15.8 mmol) are added thereto. After 12 hours' stirring at RT with the exclusion of light, the reaction is terminated by addition of 50 ml of methanol, and the reaction mixture is concentrated to dryness. The residue is taken up in 500 ml of dichloromethane and washed twice with in each case 150 ml of aqueous, saturated NaCl solution and then once with 150 ml of water. After drying over Na₂SO₄, the solvent is removed and the residue is purified by column chromatography on silica gel (n-hexane/diethyl ether/triethylamine=40:60:1). The product-containing fractions are identified by means of TLC, combined and concentrated to dryness. The product is dried under a high vacuum. A yellowish oil is obtained, which is stored with the exclusion of light.

Yield: 8.5 g (81% of theory)

TLC (n-hexane/diethyl ether/TEA=40:60:1): R_{f} value=0.43

MS (FD): m/z 807.2

### Example 8

### Synthesis of [1-(4,4′-dimethoxytrityl)]-2-succinyl-3-D,L-α-tocopherylglycerol (Toc4)

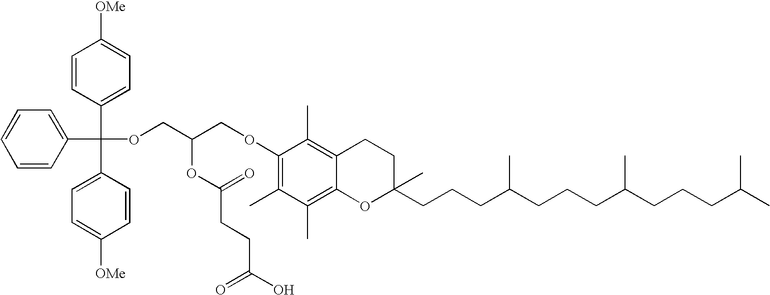

Procedure: 1.45 g of Toc3 (1.8 mmol) are dissolved twice, for drying, in 5 ml of absolute pyridine each time, which is distilled off again azeotropically. When the starting material has been dissolved in 8 ml of absolute pyridine, 140 mg of DMAP (1.08 mmol) and 194.4 mg of succinic anhydride (1.8 mmol) are added thereto in an argon countercurrent. Stirring is then carried out for 18 hours at RT with the exclusion of light. For working up, 45 ml of DCM are added to the reaction solution, and washing is carried out four times with 50 ml of water each time. After drying over sodium sulfate, the solvent is removed and the crude product is purified by column chromatography on silica gel with ethyl acetate/methanol/NH₃ (25% in H₂O)=5:1:1. The product-containing fractions are identified by means of TLC, combined and concentrated to dryness. After drying under a high vacuum, a brownish, viscous oil is obtained, which is cooled and stored with the exclusion of light.

Yield: 1.35 g (82.8% of theory)

TLC (EtOAc/NH₃/MeOH=5:1:1): R_{f} value=0.30

MS (FD): m/z 906.2 (M⁺), 604.2 (M⁺-DMT)

### Example 9

### Synthesis of D,L-α-tocopheryl-β-cyanoethyl-N,N-diisopropyl-phosphoramidite

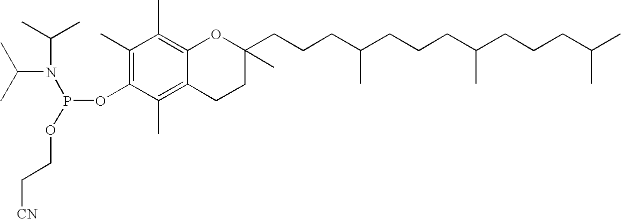

Procedure: 2×25 ml of pyridine are added to 5 g of D,L-α-tocopherol (11.6 mmol), and the mixture is dried by azeotropic entrainment. The starting material is dissolved in 40 ml of DCM_{abs}. In an argon counter-current, 7.9 ml of DIPEA_{abs} (46.4 mmol) and 2.5 ml of 2-cyanoethyl-N,N-diisopropylphosphine chloride (11 mmol) are slowly added dropwise. When the reaction mixture has been stirred for 1 hour at RT with the exclusion of light, it is diluted with 100 ml of ethyl acetate/TEA (20:1) and washed twice with 25 ml of 10% NaHCO₃ and twice with saturated NaCl solution. The organic phase is then dried over Na₂SO₄ and the solvent is removed in vacuo. The crude product is purified by column chromatography on silica gel with ethyl acetate.

Yield: 5.62 g (81% of theory)

TLC (EtOAc): R_{f} value=0.75

MS (FD): m/z 630.1 (M⁺)

³¹P-NMR: δ 152.24

### Example 10

### Synthesis of [1-(4,4′-dimethoxytrityl)]-(3-D,L-α-tocopheryl)-glycerol-2-phosphoramidite

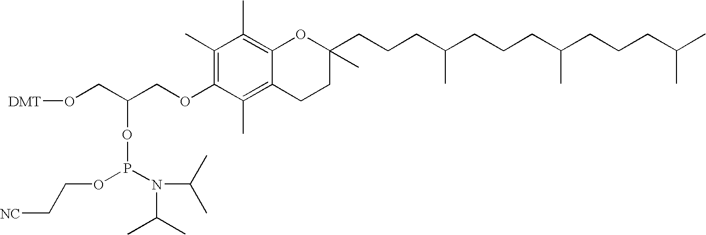

Procedure: 1 g of Toc3 (1.24 mmol) is dissolved twice, for drying, in 10 ml of absolute pyridine each time, which is distilled off again azeotropically. The starting material is then dissolved in 20 ml of DCM_{abs}, and 0.84 ml of DIPEA_{abs} (4.96 mmol) are added dropwise under an argon counter-current. 0.27 ml of 2-cyanoethyl-N,N-diisopropylphosphine chloride (1.19 mmol) are then slowly added dropwise in an argon counter-current. The reaction mixture is stirred for 1 hour at RT, before the solution is diluted with 50 ml of ethyl acetate/TEA (20:1). The organic phase is washed twice with 15 ml of a 10% NaHCO₃ solution and twice with a saturated NaCl solution and then dried over Na₂SO₄. The solvent is removed and the crude product is purified by column chromatography on silica gel with ethyl acetate/TEA (99:1). The product-containing fractions are identified by means of TLC, combined and concentrated to dryness. After drying under a high vacuum, a yellowish-brown, very viscous oil is obtained, which is cooled and stored with the exclusion of light.

Yield: 0.76 g (63.4% of theory)

TLC (EtOAc, 1% TEA): R_{f} value=0.68

MS (FD): m/z 1006.4 (M⁺), 953.6 (M⁺-cyanoethyl), 651.4 (M⁺-DMT,-cyanoethyl), 603.1 (M⁺-DMT,-diisopropylamine), 303.1 (DMT⁺)

³¹P-NMR: δ 150.5

### Example 11

### Synthesis of 1-hexadecyl-2,3-isopropylideneglycerol (Pam1)

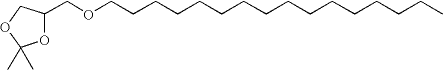

Procedure: 0.11 mol of sodium hydride (2.42 g) is added in portions, under an argon counter-current, to 0.1 mol of D,L-α,β-isopropylidene-glycerol (12.4 ml) in 500 ml of THF_{abs}. After 12 hours' stirring at RT, 0.11 mol of 1-bromohexadecane (33.6 ml) in 80 ml of THF_{abs} is added dropwise to the resulting alcoholate. After addition of 0.5 mmol of tetrabutylammonium iodide as catalyst, the mixture is heated at boiling point for 12 hours. After cooling of the reaction mixture, the resulting sodium bromide is filtered off and the filtrate is concentrated to dryness. The residue is taken up in diethyl ether and the ether phase is extracted by shaking three times with H₂O. After drying of the organic phase over Na₂SO₄, the mixture is concentrated to dryness and the residue is purified by column chromatography on silica gel (EtOAc/n-hexane=1:9) (S. Czernecki, C. Georgoulis, C. Provelenghiou Tetrahedron Lett. 39 (1976) 3535).

Yield: 18.5 g (52% of theory)

TLC (EtOAc/n-hexane=1:9): R_{f} value=0.47

MS (FD): m/z 357.6 (M⁺+1)

¹H-NMR (CDCl₃): δ 0.80 (t), 1.18 (s), 1.35 (s), 1.37 (s), 3.30-3.48 (m), 3.63-3.69 (dd), 3.96-4.01 (dd), 4.19 (q)

¹³C-NMR (CDCl₃): δ 14.1; 22.7; 25.4; 26.1; 26.8; 29.4; 29.6; 29.7; 31.9 (alkyl chain), 67.0 (alkyl-C—O—), 71.8; 71.9; 74.7 (glycerol-C), 109.3 (ketyl-C)

### Example 12

### Synthesis of 1-hexadecylglycerol (Pam2)

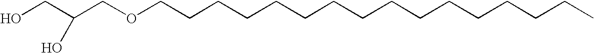

Procedure: 18.5 g of Pam1 (52 mmol) are stirred in 300 ml of acetic acid (65%) for 24 hours at 40° C. The white precipitate is filtered off and concentrated to dryness several times with n-hexane. For purification, the product is suspended three times in n-hexane and filtered off. Starting material that has not been deprotected, unlike the product, is soluble in n-hexane and can accordingly be separated off. The residue that remains is dried in vacuo. The combined filtrates are likewise concentrated to dryness and again subjected to the separation procedure (H. Paulsen, E. Meinjohanns, F. Reck, I. Brockhausen Liebigs Ann. Chem. (1993) 721).

Yield: 14.6 g (88% of theory)

TLC (EtOAc/n-hexane=1:1): R_{f} value=0.2

MS (FD): m/z 317.4 (M⁺+1)

¹H-NMR (CDCl₃): δ 0.86 (t), 1.23 (s), 3.30-3.48 (m), 3.41-3.49 (m), 3.55-3.75 (m), 3.78-3.9 (m)

¹³C-NMR (CDCl₃): δ 14.1; 22.7; 25.4; 26.1; 29.4; 29.5; 29.6; 31.9 (alkyl chain), 70.5 (alkyl-C—O—), 64.2; 71.8; 72.4 (glycerol-C)

### Example 13

### Synthesis of [1-(4,4′-dimethoxytrityl)]-3-hexadecylglycerol (Pam3)

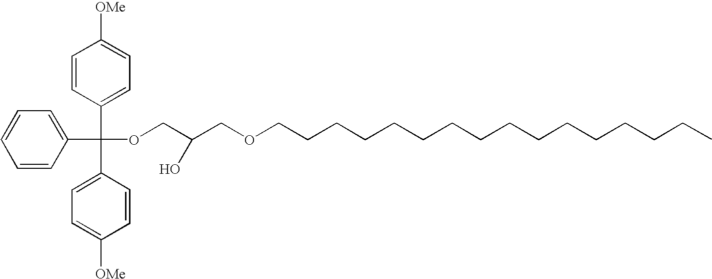

Procedure: For drying, 10 mmol of Pam2 (3.16 g) are dissolved in 15 ml of absolute pyridine and the solvent is removed again. This procedure is repeated. 12 mmol (4.06 g) of dimethoxytrityl chloride (dissolved in 50 ml of pyridine) are slowly added dropwise to a solution of the diol in 100 ml of absolute pyridine, and stirring is carried out for 24 hours at RT. The reaction is then terminated with 5 ml of methanol and then the reaction mixture is concentrated to dryness. Final traces of pyridine are removed by azeotropic entrainment with toluene. The residue is taken up in 300 ml of DCM, washed with saturated aqueous KCl solution and H₂O and dried over Na₂SO₄. After removal of the solvent, the residue is chromatographed on silica gel (n-hexane/diethyl ether/TEA=40:60:1) (R. A. Jones “Oligonucleotide Synthesis: A Practical Approach” ed. M. J. Gait, IRL Press (1984) 23).

Yield: 4.9 g (79.3% of theory)

TLC (n-hexane/diethyl ether/TEA=40:60:1): R_{f} value=0.42

MS (FD): m/z 618.2 (M⁺), 303 (DMT⁺)

¹H-NMR (CDCl₃): δ 0.80 (t), 1.20 (s), 1.96 (s), 2.36 (d), 2.72 (s), 3.27 (d), 3.32-3.48 (m), 3.75 (m), 6.61-6.76 (m), 7.08-7.37 (m)

### Example 14

### Synthesis of [1-(4,4′-dimethoxytrityl)]-2-succinyl-3-hexadecylglycerol (Pam4)

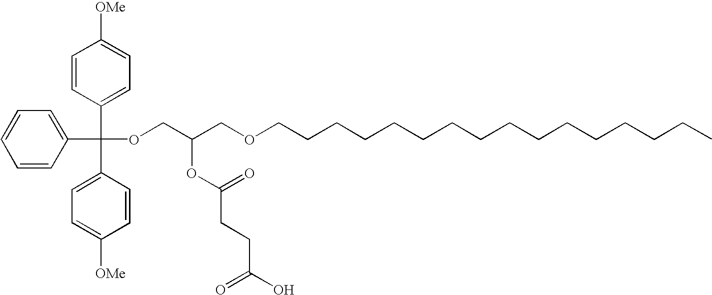

Procedure: 1.26 mmol of Pam3 (0.78 g) are dried twice with pyridine. 0.76 mmol of DMAP (92 mg) and 1.26 mmol of succinic anhydride (126 mg) are added to a solution of the alcohol in 5 ml of absolute pyridine. After 12 hours' stirring at RT, the reaction solution is taken up in 30 ml of DCM, washed twice with in each case 30 ml of water and dried over Na₂SO₄. After removal of the solvent, the residue is chromatographed on silica gel (ethyl acetate/methanol/NH₃ (25% in H₂O)=5:1:1) (D. W. Will, T. Brown Tetrahedron Lett. 33 (1992) 2729.).

Yield: 0.6 g (66.3%)

TLC (EtOAc/MeOH/NH₃(H₂O)=5:1:1): R_{f} value=0.32

MS (FD): m/z 718 (M⁺), 303 (DMT⁺), 1020.7 (M+DMT)⁺

### Example 15

Stimulation of human cells with an adjuvant according to the invention in the form of a lipid-modified nucleic acid

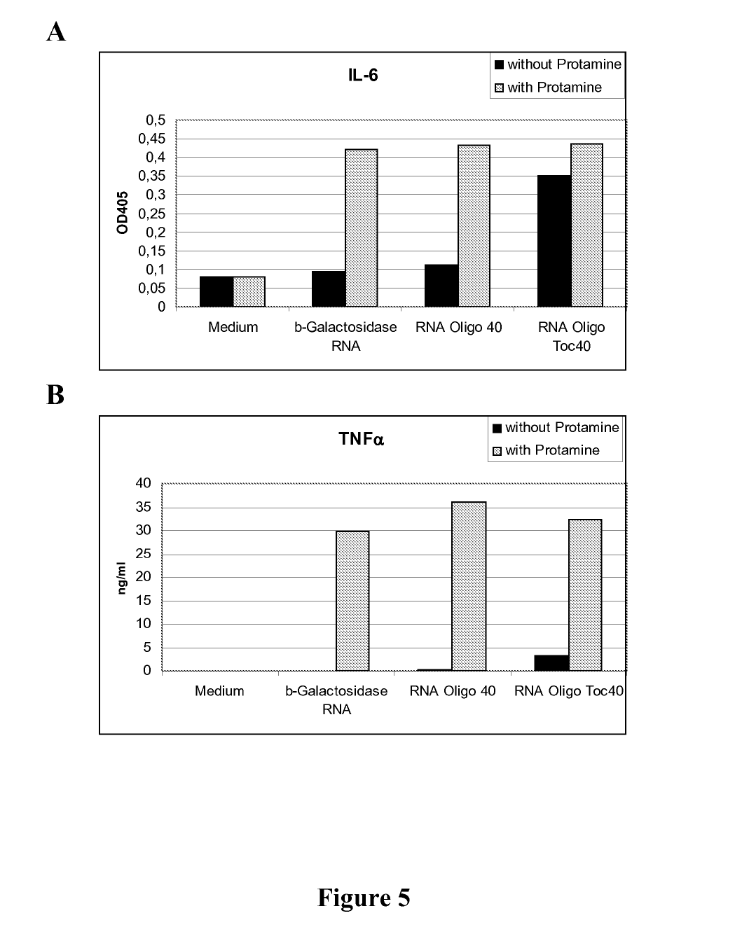

In order to determine the immunogenic activity of adjuvants according to the invention, such adjuvants in the form of a lipid-modified nucleic acid containing a sequence according to SEQ ID NO: 40, 41 or 42 were co-incubated with human cells. To this end, human PBMC cells, for example, containing RNA were co-incubated for 16 hours in X-vivo15 medium (BioWhittaker), enriched with 2 mM L-glutamine (BioWhittaker), 10 U/ml penicillin (BioWhittaker) and 10 μg/ml streptomycin, with 10 μg/ml of RNA (mRNA coding for β-galactosidase or phosphorothioate RNA oligonucleotide 40 5′ GCCCGUCUGUUGUGUGACUC (SEQ ID NO: 40)) and optionally with 10 μg/ml protamine. The supernatants were removed and analysed by means of ELISA. Particularly in the case of the release of cytokines (IL-6) it is to be observed that the adjuvants according to the invention without the addition of protamine exhibit a more than 5-fold increase in cytokine release (IL-6) as compared with the medium and, with the addition of protamine, a slightly improved release of IL-6 as compared with β-galactosidase and RNA oligo 40 alone (SEQ ID NO: 40) (see FIG. 5A). When determining the TNFα release, a marked stimulation of the immune system can be detected, which is at least equal to that of β-galactosidase or RNA (see FIG. 5B).

In a further experiment, the release of TNF-α by human PBMC cells was determined after stimulation with RNA oligonucleotides used according to the invention and also adjuvants according to the invention.

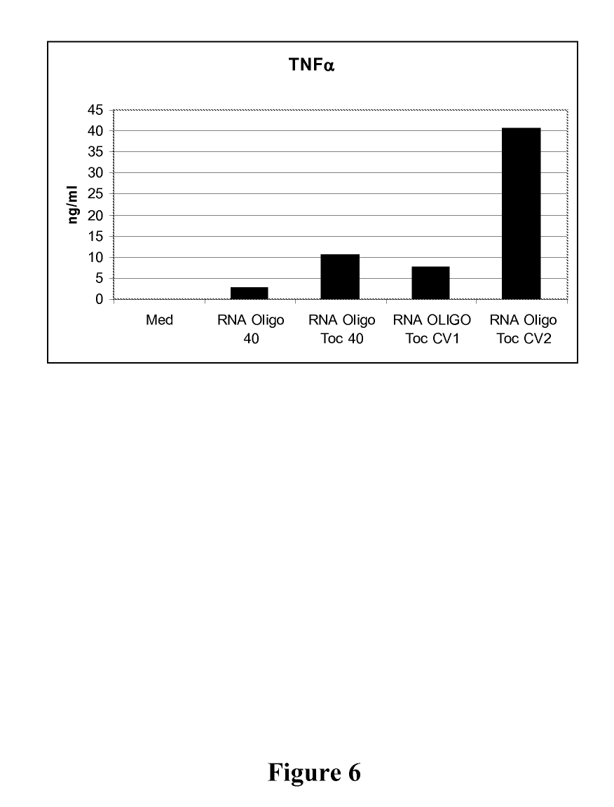

To that end, human PBMC cells were co-incubated for 16 hours with 10 μg/ml of RNA oligonucleotides in X-vivo15 medium (BioWhittaker), enriched with 2 mM L-glutamine (BioWhittaker), 10 U/ml penicillin (BioWhittaker) and 10 μg/ml streptomycin. The RNA oligos used here by way of example for the lipid modification carried the following sequences: RNA 40: 5′ GCCCGUCUGUUGUGUGACUC (SEQ ID NO: 40), RNA CV1: GGUAAGUGUAAGGUGUAAGG (SEQ ID NO: 41), RNA CV2: AAUGGAUAUGGAAUAUGGAA (SEQ ID NO: 42). The supernatants were removed and analysed by means of ELISA. It is clear that each adjuvant in the form of a lipid-modified nucleic acid having, for example, a sequence according to SEQ ID NO: 40, 41 or 42 exhibits a markedly improved release of TNF-α as compared with, for example, an unmodified RNA oligonucleotide having the sequence according to SEQ ID NO: 40 (RNA oligo 40) (or 41 or 42) and accordingly exhibits markedly improved immune stimulation. The best results, with a more than 10-fold increase in immune stimulation as compared with the unmodified RNA oligonucleotide, were obtained with a lipid-modified sequence according to SEQ ID NO: 42 (see FIG. 6).

The immune-stimulating adjuvant according to the invention in the form of a lipid-modified nucleic acid, in particular in the form of a 5′- and/or 3′-lipid-modified nucleic acid, on the one hand permits stabilisation and better cell permeability of (pharmacological) active ingredients and also better distribution of the active ingredients within the cell. In addition, it is of critical importance that the immune-stimulating adjuvant according to the invention is itself able to bring about an increase in the immune reaction as a biological activity. This can happen on the one hand by supporting the actual anti-sense mechanism, for example by better binding of the (lipid-modified) nucleic acids used, owing to intercalation; on the other hand, an independent activity of the immune-stimulating adjuvant according to the invention for increasing stimulation is also conceivable. For example, according to the invention, a 3′-cholesterol-modified phosphodiester oligonucleotide has been disclosed as an immune-stimulating adjuvant that is able to have a cytotoxic effect on specific tumour cells. Although the 3′-cholesterol-modified phosphodiester oligonucleotide according to the invention is also able to act sequence-specifically, it cannot act through anti-sense effects. Also, the receptor-mediated endocytosis described for cholesterol does not make a substantial contribution to the unusual immune-stimulating activity that has been found. This surprising effect is therefore based substantially on an immune-stimulating action of the lipid-modified nucleic acid of the adjuvant according to the invention that is used, the nature of the 3′ or 5′ modification (lipid modification) playing an important role. In summary, the immune-stimulating adjuvant according to the invention therefore exhibits an advantageous immune-stimulating action and at the same time increases the (cell) permeability of any active ingredients that may additionally be present.

While the invention has been described in detail, and with reference to specific embodiments thereof, it will be apparent to one of ordinary skill in the art that various changes and modifications can be made therein without departing from the spirit and scope thereof and such changes and modifications may be practiced within the scope of the appended claims. All patents and publications herein are incorporated by reference to the same extent as if each individual publication was specifically and individually indicated to be incorporated by reference in their entirety.

What is claimed is:

1. An immune-stimulating adjuvant comprising a lipid-modified nucleic acid.

2. An immune-stimulating adjuvant according to claim 1, wherein the lipid-modified nucleic acid comprises a nucleic acid covalently attached to a linker and a lipid covalently attached to the linker.

3. An immune-stimulating adjuvant according to claim 1, wherein the lipid-modified nucleic acid comprises at least one nucleic acid and at least one bifunctional lipid covalently linked to the nucleic acid.

4. An immune-stimulating adjuvant according to claim 1, wherein the lipid-modified nucleic acid comprises a nucleic acid, at least one linker covalently linked to the nucleic acid, at least one lipid covalently linked to the linker, and at least one bifunctional lipid covalently linked to the nucleic acid.

5. An immune-stimulating adjuvant according to claim 1, wherein the lipid-modified nucleic comprises a nucleic acid covalently attached to a linker and contains 3 to 8 lipids per nucleic acid, wherein at least one lipid is covalently linked with the linker.

6. An immune-stimulating adjuvant according to claim 1, wherein the lipid-modified nucleic comprises a nucleic acid covalently attached to a linker and contains 3 to 8 lipids per nucleic acid and wherein all of the lipids are covalently linked with the linker.

7. An immune-stimulating adjuvant according to claim 1, wherein the lipid-modified nucleic contains 3 to 8 lipids per nucleic acid and wherein the lipids are covalently linked directly with the nucleic acid.

8. An immune-stimulating adjuvant according to claim 1, wherein the nucleic acid of the lipid-modified nucleic acid is selected from a group consisting of RNA, DNA, an RNA oligonucleotide, a DNA oligonucleotide, an RNA homopolymer, a DNA homopolymer or a CpG nucleic acid.

9. An immune-stimulating adjuvant according to claim 1, wherein the nucleic acid of the lipid-modified nucleic acid is selected from a group consisting of a single-stranded nucleic acid, a double-stranded nucleic acid, a homoduplex nucleic acid, a heteroduplex nucleic acid, a linear nucleic acid, and a circular nucleic acid.

10. An immune-stimulating adjuvant according to claim 1, wherein the nucleic acid of the lipid-modified nucleic acid has a length selected from the group consisting of from approximately 2 to approximately 1000 nucleotides, from approximately 5 to approximately 200 nucleotides, from approximately 6 to approximately 100 nucleotides, from approximately 6 to approximately 40 nucleotides, and from approximately 6 to approximately 31 nucleotides.

11. An immune-stimulating adjuvant according to claim 1, wherein the nucleic acid comprises a sequence selected from the group consisting of SEQ ID NOs: 1-67.

12. An immune-stimulating adjuvant according to claim 1, wherein the nucleic acid comprises a sequence that is at least 60% identical with a sequence selected from the group consisting of SEQ ID NOs: 1-67.

13. An immune-stimulating adjuvant according to claim 1, wherein at least one lipid is selected from the group consisting of vitamins, α-tocopherol (vitamin E), RRR-α-tocopherol (D-α-tocopherol), L-α-tocopherol, racemate D,L-α-tocopherol, vitamin A, derivatives of vitamin A, retinoic acid, retinol, vitamin D, derivatives of vitamin D, ergosterol precursors of vitamin D, vitamin E, derivatives of vitamin E, vitamin E succinate (VES), vitamin K, derivatives of vitamin K, quinone compounds, phytol compounds, steroids, bile acids, cholic acid, deoxycholic acid, dehydrocholic acid, cortisone, digoxygenin, testosterone, cholesterol, thiocholesterol, polyalkylene glycols, aliphatic groups, C1-C20-alkanes, C1-C20-alkenes, C1-C20-alkanols, dodecanediol, hexadecanol, undecyl radicals, phospholipids, phosphatidylglycerol, diacylphosphatidylglycerol, phosphatidylcholine, dipalmitoylphosphatidylcholine, distearoylphosphatidylcholine, phosphatidylserine, phosphatidylethanolamine, di-hexadecyl-rac-glycerol, sphingolipids, cerebrosides, gangliosides, triethylammonium 1,2-di-O-hexadecyl-rac-glycero-3-H-phosphonate, polyamines, polyalkylene glycols, polyethylene glycol (PEG), hexaethylene glycol (HEG), palmitin, palmityl radicals, octadecylamines, hexylamino-carbonyl-oxycholesterol radicals, waxes, terpenes, alicyclic hydrocarbons, saturated fatty acid radicals, or mono-unsaturated fatty acid radicals, or poly-unsaturated fatty acid radicals.

14. An immune-stimulating adjuvant according to claim 2, wherein the linker contains 2-4 reactive groups and the reactive groups are independently selected from the group consisting of a hydroxy group, an amino group and an alkoxy group.

15. An immune-stimulating adjuvant according to claim 14, wherein the linker is selected from the group consisting of glycol, glycerol, glycerol derivatives, 2-aminobutyl-1,3-propanediol, 2-aminobutyl-1,3-propanediol derivatives, a 2-aminobutyl-1,3-propanediol scaffold, pyrrolidine linkers, and pyrrolidine-containing organic molecules.

16. An immune-stimulating adjuvant according to claim 1, wherein the nucleic acid comprises a 3′ end and a 5′ end and the nucleic acid is modified with lipid at the 3′-end, the 5′-end or at both the 3′-end and the 5′-end.

17. An immune-stimulating adjuvant according to claim 2, wherein the nucleic acid comprises a 3′ end and a 5′ end and the linker is attached to the nucleic acid at the 3′-end, the 5′-end or at both the 3′-end and the 5′-end.

18. An immune-stimulating adjuvant according to claim 1, wherein the nucleic acid of the lipid-modified nucleic acid comprises at least one chemical modification.

19. An immune-stimulating adjuvant according to claim 1, wherein the nucleic acid is RNA comprising a 5′-end and a 3′-end and comprises a 5′-end a cap structure, a 3′-end poly-A tail, or both a 5′-end a cap structure and a 3′-end poly-A tail.

20. An immune-stimulating adjuvant according to claim 1 and further comprising an adjuvant selected from the group consisting of aluminium hydroxide, complete Freund's adjuvant, incomplete Freund's adjuvant, stabilising cationic peptides, polypeptides, protamine, nucleoline, spermine, spermidine, cationic polysaccharides, chitosan, TDM, MDP, muramyl dipeptide, alum solution, pluronics, lipopeptides, and Pam3Cys.

21. A pharmaceutical composition comprising an immune-stimulating adjuvant comprising a lipid-modified nucleic acid and at least one active ingredient.

22. A pharmaceutical composition according to claim 21, further comprising at least one ingredient selected from the group consisting of pharmaceutically acceptable carriers, pharmaceutically acceptable additives and adjuvants.

23. A pharmaceutical composition according to claim 22, wherein the active ingredient is selected from peptides, proteins, nucleic acids, low molecular weight organic or inorganic compounds having a molecular weight less than 5000, sugars, antigens, antibodies, and therapeutic agents.

24. A pharmaceutical composition according to claim 23, further comprising an adjuvant selected from the group consisting of aluminium hydroxide, complete Freund's adjuvant, incomplete Freund's adjuvant, stabilising cationic peptides, polypeptides, protamine, nucleoline, spermine, spermidine, cationic polysaccharides, chitosan, TDM, MDP, muramyl dipeptide, alum solution, pluronics, lipopeptides, and Pam3Cys.

25. A pharmaceutical composition according to claim 21, wherein the pharmaceutical composition is a vaccine.

26. A method of treating a disease in a subject in need thereof, comprising:
administering an immune-stimulating adjuvant according to claim 1.

27. A method according to claim 26, further comprising administering a therapeutic agent.

28. A method according to claim 26, wherein the disease is cancer.

29. A method according to claim 26 wherein the disease is an infectious disease.

30. A method according to claim 26, wherein the disease is selected from the group consisting of colon carcinomas, melanomas, renal carcinomas, lymphomas, acute myeloid leukaemia (AML), acute lymphoid leukaemia (ALL), chronic myeloid leukaemia (CML), chronic lymphocytic leukaemia (CLL), gastrointestinal tumours, pulmonary carcinomas, gliomas, thyroid tumours, mammary carcinomas, prostate tumours, hepatomas, virus-induced tumours, papilloma virus-induced carcinomas, cervical carcinoma, adenocarcinomas, herpes virus-induced tumours, Burkitt's lymphoma, EBV-induced B-cell lymphoma, heptatitis B-induced tumours, hepatocell carcinoma, HTLV-1-induced lymphomas, HTLV-2-induced lymphomas, acoustic neuromas, cervical cancer, lung cancer, pharyngeal cancer, anal carcinomas, glioblastomas, lymphomas, rectal carcinomas, astrocytomas, brain tumours, stomach cancer, retinoblastomas, basaliomas, brain metastases, medulloblastomas, vaginal cancer, pancreatic cancer, testicular cancer, melanomas, thyroidal carcinomas, bladder cancer, Hodgkin's syndrome, meningiomas, Schneeberger disease, bronchial carcinomas, hypophysis tumour, Mycosis fungoides, esophageal cancer, breast cancer, carcinoids, neurinomas, spinaliomas, laryngeal cancer, renal cancer, thymomas, corpus carcinomas, bone cancer, non-Hodgkin's lymphomas, urethral cancer, CUP syndrome, head tumors, neck tumours, oligodendrogliomas, vulval cancer, intestinal cancer, colon carcinomas, esophageal carcinomas, warts, tumours of the small intestine, craniopharyngeomas, ovarian carcinomas, genital tumours, ovarian cancer, liver cancer, pancreatic carcinomas, cervical carcinomas, endometrial carcinomas, liver metastases, penile cancer, tongue cancer, gall bladder cancer, leukaemia, plasmocytomas, uterine cancer, lid tumour and prostate cancer.

31. A method according to claim 29, wherein the infectious disease is selected from the group consisting of influenza, malaria, SARS, yellow fever, AIDS, Lyme borreliosis, Leishmaniasis, anthrax, meningitis, viral infectious diseases, AIDS, Condyloma acuminata, hollow warts, Dengue fever, three-day fever, Ebola virus, cold, early summer meningoencephalitis (FSME), flu, shingles, hepatitis, herpes simplex type I, herpes simplex type II, Herpes zoster, influenza, Japanese encephalitis, Lassa fever, Marburg virus, measles, foot-and-mouth disease, mononucleosis, mumps, Norwalk virus infection, Pfeiffer's glandular fever, smallpox, polio, pseudo-croup, German measles, rabies, warts, West Nile fever, chickenpox, cytomegalic virus (CMV), bacterial infectious diseases, miscarriage, prostate inflammation, anthrax, appendicitis, borreliosis, botulism, Camphylobacter, Chlamydia trachomatis, inflammation of the urethra, conjunctivitis, cholera, diphtheria, donavanosis, epiglottitis, typhus fever, gas gangrene, gonorrhoea, rabbit fever, Heliobacter pylori, whooping cough, climatic bubo, osteomyelitis, Legionnaire's disease, leprosy, listeriosis, pneumonia, meningitis, bacterial meningitis, anthrax, otitis media, Mycoplasma hominis, neonatal sepsis, Chorioamnionitis, noma, paratyphus, plague, Reiter's syndrome, Rocky Mountain spotted fever, Salmonella paratyphus, Salmonella typhus, scarlet fever, syphilis, tetanus, tripper, tsutsugamushi disease, tuberculosis, typhus, vaginitis, colpitis, soft chancre, parasitic infectious diseases, protozoal infectious diseases, fungal infectious diseases, amoebiasis, bilharziosis, Chagas disease, athlete's foot, yeast fungus spots, scabies, malaria, onchocercosis (river blindness), toxoplasmosis, trichomoniasis, trypanosomiasis (sleeping sickness), visceral Leishmaniosis, nappy dermatitis, schistosomiasis, fish poisoning (Ciguatera), candidosis, cutaneous Leishmaniosis, lambliasis (giardiasis), sleeping sickness, infectious diseases caused by Echinococcus, infectious diseases caused by fish tapeworm, infectious diseases caused by fox tapeworm, infectious diseases caused by canine tapeworm, infectious diseases caused by lice, infectious diseases caused by bovine tapeworm, infectious diseases caused by porcine tapeworm and infectious diseases caused by miniature tapeworm.

32. A kit containing an immune-stimulating adjuvant according to claim 1 and comprising technical instructions with information on the administration and dosage of the immune-stimulating adjuvant.

33. A kit containing a pharmaceutical composition according to claim 21 and comprising technical instructions with information on the administration and dosage of the pharmaceutical composition.
